(19) United States
(12) Patent Application Publication
(10) Pub. No.: US 2021/0237078 A1
(43) Pub. Date: Aug. 5, 2021

(54) SYSTEMS, DEVICES AND METHODS ASSOCIATED WITH MICROFLUIDIC SYSTEMS

(71) Applicants: Miriam Cather SIMPSON, Auckland (NZ); Peter Anthony Greenwood HOSKING, Auckland (NZ); Simon Andrew ASHFORTH, Kumeu (NZ); Claude AGUERGARAY, Auckland (NZ); Engender Technologies Limited, Hamilton (NZ)

(72) Inventors: Miriam Cather SIMPSON, Auckland (NZ); Peter Anthony Greenwood HOSKING, Auckland (NZ); Simon Andrew ASHFORTH, Kumeu (NZ); Claude AGUERGARAY, Auckland (NZ); Michael Ronald KEOUGH, Auckland (NZ)

(21) Appl. No.: 17/050,417

(22) PCT Filed: Apr. 25, 2019
(86) PCT No.: PCT/US2019/029238

§ 371 Date: Oct. 23, 2020

Related U.S. Application Data
(60) Provisional application No. 62/690,869, filed on Jun. 27, 2018.
(60) Provisional application No. 62/688,503, filed on Jun. 22, 2018.
(60) Provisional application No. 62/662,609, filed on Apr. 25, 2018.

(51) Int. Cl. B01L 3/00

(52) U.S. Cl. CPC B01L 3/502761; B01L 2200/0636; B01L 2200/0652

(57) ABSTRACT

The present application discloses a plurality of embodiments and associated inventions, with respect to microfluidic systems for at least one of identifying, imaging, orientating, and sorting particles, in particular, biological cells, and more particularly, X and Y sperm cells. In some embodiments, a module system with functional connectors is provided, each module being connected by a connector that can provide additional functionality aside from enabling fluid flow between modules. The present disclosure also is directed to microfluidic systems which include particle delivery tubes configured to orient particles (e.g., X and Y sperm cells), as well as microfluidic systems for generating a static, spatial patterns within the microfluidic channel.

### RELATED APPLICATIONS

The present disclosure claims benefit of and priority to U.S. provisional patent application nos. 62/662,609, entitled, “MICROFLUIDIC CHIP BLOCK SYSTEM AND METHODS OF USING SAME,” filed Apr. 25, 2018, 62/688,503, entitled, “MICROFLUIDIC SYSTEM AND METHODS FOR ORIENTING ASYMMETRIC PARTICLES,” filed Jun. 22, 2018, and 62/690,869, entitled, “SYSTEMS, APPARATUSES, DEVICES AND METHODS FOR SORTING AND/OR ORIENTING PARTICLES IN A MICROFLUIDIC SYSTEM,” filed Jun. 27, 2018. Each of the foregoing disclosures are incorporated herein by reference in their entirety.

### BACKGROUND OF THE DISCLOSURE

While considerable progress has been made in the design and use of microfluidic systems for manipulating particles of a sample (in particular, for isolating a given type of particles from other particles), a need remains for less expensive and smaller microfluidic systems, in particular, microfluidic systems which are portable, e.g., easily moved between sites of operation, and for systems that provide for multiple operations that involve more functionalities than fluid flow and mixing, and permit enhanced manipulation of a fluid sample and/or data obtained, therefrom.

### SUMMARY OF SOME OF THE EMBODIMENTS OF THE DISCLOSURE

### Modular & Functional Connector Aspects

In some embodiments of the present disclosure, a microfluidic system is provided comprising at least two modules/blocks/stages (such terms being used interchangeably throughout), where the at least two modules are attached via a functional connector. In some embodiments, the modular microfluidic system comprises at least two modules connected by a transparent capillary to allow light into and out of the microfluidic system

Thus, in some embodiments, the present disclosure provides a microfluidic system comprising at least two modules/blocks/stages, and in some embodiments, three (3) or more modules, where at least some (and in some embodiments, a plurality of module sets (which, in some embodiments, are adjacent), and in some embodiments, all of the module sets—which may also be adjacent) module sets/pairs are connected together via a functional connector—i.e., one that performs at least one specific function over merely flowing fluid or fluid mixtures from one module to the next or merely providing a structural connectivity between or among modules.

In some embodiments, a modular, microfluidic sorting system for sorting particles in a microfluidic system is provided and includes, a plurality of modules configured to be arranged in a plurality of configurations depending upon at least one of the number and type of modules provided, and the desired functionality of the system. The plurality of modules at least including a first module having at least one input port, a first module channel connected to the at least one input port, and at least one output port connected to the distal end of the first module channel, at least one second module with at least one input port, a second module channel connected to the at least one input port, and at least one output port connected to the distal end of the second module channel, and at least one third module with at least one input port, a third module channel connected to the at least one input port, and at least two output ports connected to the distal end of the third module channel. The system further includes at least one first connector connecting the at least one first module and the at least one second module, and at least one second connector connecting the at least one second module and the at least one third module. Each connector includes a lumen surrounded by a wall, where the lumen is configured to flow at least particles contained in a fluid therethrough and between connected modules. Each connector also includes a first end in fluid communication with an output port of one of the connected modules, a second end in fluid communication with the input port of a remaining one of the connected modules, and at least one of the connectors comprises a sorting connector configured to effect a sorting function for the plurality of particles flowing therein.

Such embodiments may include at least one of (and in some embodiments, preferably a plurality of, and in some further embodiments, preferably include all of) the following features, structure, functionality, steps, and/or clarifications, yielding yet further embodiments of the present disclosure (the following can be mixed and matched to obtain desired module and/or system functionality as a whole):
at least a first portion and/or another portion of the wall of at least one of the connectors is configured to at least one of receive light into the lumen and transmit light out of the lumen, or at least a first portion and/or another portion of the wall of the sorting connector is configured to at least one of receive and transmit light through the wall;
at least the first portion and/or another portion comprises glass, quartz, or a polymer;
a source for each module input port, where the source may be connected to a respective module input port via an associated source tube and/or connector;
at least one of the module channels, the connectors, and/or the source tubes comprises a capillary tube;
at least one of the module channels passes through a respective module;
at least one of the one input sources for at least one of the modules is configured to introduce a fluid into a respective module channel as a sheath flow;
at least one of the at least one input sources for at least one of the modules is configured to introduce a flow of particles into a respective module channel;
the at least one of receiving light into the lumen and transmitting light out of the lumen is configured for at least one of: receiving light so as to induce one or more fluorescence signals of material flowing within the lumen of the connector, transmitting one or more fluorescence signals generated by material flowing within the lumen of the connector through the wall, receiving light so as to induce a force or a torque upon material flowing within the lumen of the connector, transmitting light through the wall so as to induce one or more scattering signals by material flowing within the lumen of the connector, transmitting scattered light signals generated by material flowing within the lumen of the connector, transmitting light so as to illuminate at least one of the particles flowing within the lumen of the connector for imaging of the at least one of the particles, and transmitting light reflected off material flowing within the lumen for imaging the material;
the third module comprises a collection module;
   the at least two output ports of the collection module may be configured to collect material passed to the collection module from the at least one second module; and/or
   a first of the at least two output ports of the collection module collect particles of interest received from the second module, and a second of the at least two outputs of the collection module collects waste.
the system is configured to provide hydrodynamic flow in multiple dimensions, where the dimensions comprise three-dimensions;
each module and at least one of the connectors are configured with at least one, respective specific functionality for the microfluidic sorting system;
   the at least one specific functionality may be selected from the group consisting of: particle entry, particle sheathing, particle focusing, particle orienting, particle detecting, particle discrimination, particle sorting, and at least one of sample and particle collection;
each module may comprise a plurality of functions;
each module may comprise a plurality of sides, where input ports and output ports are configured for arrangement on any side;
all of the input ports are arranged on a first side and all of the output ports are arranged on a second side;
and
one or more of the input ports are arranged on a first side and one or more of the output ports are arranged on a second side, where at least one input port and at least one output port may be arranged on a first side, and at least one input port and at least one output port may be arranged on a second side.

In some embodiments, a microfluidic sorting method for sorting particles in a microfluidic system is provided, and includes, providing a modular, microfluidic sorting system for sorting particles in a microfluidic system, according to any of the disclosed embodiments, directing a sheath fluid flow from at least one first input source into at least one input port of at least one first module, directing a plurality of particles in a fluid from at least one second input source into at least one of the module channels within the sheath flow to create a particle flow, first passing the particle flow from one of the modules to another via at least one of the connectors, at least one of:
directing light into the at least one connector so as to illuminate material inside the connector;
at least one of monitoring and imaging light signals generated by the material within the lumen through the wall; and
directing light into at least one connector so as to induce at least one of a force and a torque on material flowing inside the connector;

The method also includes, passing of the particle flow (e.g., second passing relative to the first passing recited above) from one module into another module via at least one other connector, and at least one of:
ultimately directing material of interest received from at least one module through at least one connector to a collecting module and into a particle collection output port, and
ultimately directing waste material received from at least one module through at least one connector through a collecting module and into a waste collection output.

In some embodiments, a modular microfluidic particle method is provided, and includes interconnecting a plurality of modules configured to be interconnected in at least two arrangements, where each module and at least one connector includes at least one associated function. In some such embodiments, the associated function may be selected from the group consisting of particle entry, particle sheathing, particle focusing, particle orienting, particle detecting, particle discrimination, particle sorting and sample or particle collection.

### Particle Orientation & Delivery Tube Aspects

In some embodiments of the present disclosure, a particle orientation system (and in some embodiments, a particle orientation system which can position a particle flow and/or split and position the flow within a channel) is provided which is configured for at least positioning and/or orienting particles in a fluid flow within a microfluidic channel. The system includes at least one microfluidic channel and/or chamber configured for at least one of receiving and flowing at least a sheath fluid, and a particle orientation and delivery tube (“PODT”) configured for delivering a particle-containing fluid having at least a plurality of particles within a fluid into the sheath fluid within the microfluidic channel or chamber. At least one of the PODT, the microfluidic channel, and chamber wall includes at least one structural feature (in some embodiments, a structural feature—i.e., one effected in the material making up the component) configured to impart an orienting torque to the plurality of particles within the sheath fluid.

In some embodiments of the disclosure, a PODT configured for use in a particle orientation system is provided, where the PODT is configured to orient a plurality of particles within a fluid, and the PODT includes at least one structural feature with or on at least one of the internal surface, the external surface configured to impart a torque to the plurality of particles within the fluid.

Such embodiments (as described above, for example) may include at least one of (and in some embodiments, preferably a plurality of, and in some further embodiments, preferably include all of) the following features, structure, functionality, steps, and/or clarifications, yielding yet further embodiments of the present disclosure:
the feature comprises at least one of a chamfer, cutaway or angled surface;
each chamfer, cutaway or angled surface can be between 10 and 80 degrees from the normal to the external surface of the PODT in any direction;
a sheath fluid tube configured to direct sheath fluid into the microfluidic channel or chamber;
the PODT is inserted within at least one of the microfluidic channel or chamber, and the sheath fluid tube;
the at least one feature is configured to generate an asymmetric pattern of laminar flow of the sheath fluid and fluid that contains the plurality of particles;
the torque orients the particles at one or more stable points relative to a frame of reference comprising the microfluidic channel;
the PODT includes a distal end which projects into the microfluidic channel or chamber;
at least the distal end of the PODT is arranged at a particular location within the microfluidic channel or chamber in at least one location relative to a frame of reference comprising the microfluidic channel or chamber;
the plurality of particles can comprise asymmetric particles;
the plurality of particles comprise cells;
the plurality of particles comprise sperm;
and
the system is configured as an orientation stage within a microfluidic system, and the system can be configured to as a cell sorting system;

In some embodiments, a particle orientation method is provided which is configured for orienting a plurality of particles in a fluid contained within a microfluidic channel or chamber. The method includes providing a system or PODT according to any such embodiment disclosed herein, flowing a sheath fluid within at least one of the sheath tube and a microfluidic channel or chamber, flowing a fluid including a plurality of particles via the PODT into the sheath fluid, and orientating the plurality of particles within the fluid. Orientating is produced via the at least one structural feature included with or on at least one of the internal surface and the external surface of the POTD and the internal surface of the microfluidic channel or chamber.

In some embodiments, a particle orientation method is provided which is configured for orienting a plurality of particles in a fluid contained within a microfluidic channel. The method includes flowing a sheath fluid within at least one of a sheath tube and a microfluidic channel or chamber, flowing a fluid including a plurality of particles (in some embodiments asymmetric particles) via a PODT into the sheath fluid, and imparting a torque to the plurality of particles so as to orient the particles at one or more stable points relative to a frame of reference comprising the microfluidic channel or channel.

Such embodiments (as described above, for example) may include at least one of (and in some embodiments, preferably a plurality of, and in some further embodiments, preferably include all of) the following features, structure, functionality, steps, and/or clarifications, yielding yet further embodiments of the present disclosure:
imparting a torque to the plurality of particles is accomplished via at least one feature included with or on at least one of the internal surface, and the external surface of the POTD and the internal surface of the microfluidic channel or chamber; and
prior to flowing a fluid including a plurality of particles, in some embodiments asymmetric particles, via a PODT into the sheath fluid, the method further comprises inserting PODT within at least one of the sheath tube and microfluidic channel.

### Spatial Patterning Aspects

In some embodiments of the disclosure, a particle manipulation system for at least one of orientating and sorting a plurality of particles is provided. The system includes a microfluidic channel configured to contain a fluid flow including a plurality of particles (in some embodiments, asymmetric particles) and at least one radiation source (RS) configured to direct radiation on the plurality of particles to effect at least one of a force and torque on each particle so as to induce at least one of displacing and orienting each particle relative to an axis defined by the direction of the fluid flow along the microfluidic channel. The system also includes at least one of free-space optics, fiber-optics, and other waveguides, configured to direct the radiation onto the fluid flow.

Such embodiments (as described above, for example) may include at least one of (and in some embodiments, preferably a plurality of, and in some further embodiments, preferably include all of) the following features, structure, functionality, steps, and/or clarifications, yielding yet further embodiments of the present disclosure:
the RS comprises a laser;
the RS is configured for strobe operation;
a sensor configured to detect at least one marker of a particle, where the marker may be used to distinguish between particles, and/or the RS can be triggered by sensing of the marker of a particle;
the marker is selected from the group consisting of: fluorescence, absorption, scatter and imaging;
one or more RSs can generate one or more static, spatial patterns within the microfluidic channel;
   the spatial pattern can be generated via either a single beam generated by the at least one RS, or by multiple beams relative to one another by two or more RSs;
   the spatial pattern comprises a 2D pattern relative to a frame of reference of the microfluidic channel;
   the spatial pattern comprises a 3D pattern relative to a frame of reference of the microfluidic channel;
   the spatial pattern can be based at least upon a position(s) of a beam(s) of the RS relative to the frame of reference of the microfluidic channel;
   the spatial pattern can be based at least upon an alignment of the propagation direction of a beam of the at least one RS with the axis of flow of the microfluidic channel;
   the spatial pattern can be based at least upon a position of a focal point of a beam produced by the at least one RS relative to the frame of reference of the microfluidic channel;
   the spatial pattern is based at least upon the spatial shape of one or more beams generated by the at least one RS;
      the spatial shape is selected from the group consisting of: Gaussian, Bessel, vortex top hat, flat top, Airy, Azimuthal, and Super-Gaussian;
   and
   the spatial pattern is based at least upon one or more of: an intensity of one or more of the beams of the at least one RS, the wavelength of one or more of the beams of the at least one RS, the polarization of one or more of the beam of the at least one RS, and any combination of the position, focal point position, spatial shape, intensity, wavelength and polarization, of one or more of the beams;
a controller and/or dynamic adjustment means configured to control and/or dynamically control the at least one RS;
   the dynamic adjustment means can dynamically control the at least one RS in real-time;
   the controller can be configured to control the dynamic adjustment means;
   the controller and/or the dynamic adjustment means can be configured to adapt characteristics of the at least one RS so as to create a dynamic, spatial, and temporal pattern during a single sorting event;
   at least one of the controller and the dynamic adjustment means can be configured to adapt to a particle orienting event;
   the at least one RS can comprise a plurality of RSs, where at least one of the controller and the dynamic adjustment means can independently controls each RS;
   the dynamic adjustment means can be configure to adjust at least one of:
      the position of a respective beam of the at least one RS relative to a frame of reference of the microfluidic channel;
      the alignment of a propagation direction of a respective beam of the at least one RS with the axis of flow of the microfluidic channel;
      the focal point of a respective beam of the at least one RS relative to a frame of reference of the microfluidic channel;
      the spatial shape of a respective beam of the at least one RS;
      the intensity of a respective beam of the at least one RS;
      the wavelength of a respective beam of the at least one RS; and
      the polarisation of a respective beam of the at least one RS;
   dynamic adjustment can be configured to adjust a beam produced by the at least one RS by effecting adjustment at the RS, or adjusting the beam at any point along an optical pathway from the output of the RS to an interaction of the beam with the particle;
   the dynamic adjustment means adjusts the at least one RS and/or a respective beam of the at least one RS via at least one of mechanical, electrical, optical, piezo-electrical, magnetic, acoustic, and pneumatic means;
and
the/a sensor which can be an imager configured to capture image information of each of the plurality of particles.

Still other embodiments of the present disclosure are directed toward combinations of the above noted embodiments, as well as one or more structures, features, steps, and functionality thereof, including combinations of two or more such structures, features, steps, and functionality thereof. Thus, such further embodiments include any of:
a system according to any of the embodiments disclosed herein;
a system comprising any one or more of the system embodiments disclosed and/or claimed herein, and/or further comprising one or more features, elements, and/or functionality of any one and/or another of the system embodiments disclosed herein;
a device comprising any one or more of the devices, or device components of system embodiments, disclosed and/or claimed herein, and/or further comprising one or more features, elements, and/or functionality of any one and/or another of the device and/or system embodiments disclosed herein;
a method according to any of the embodiments disclosed herein; and
a method comprising any one or more of the method embodiments disclosed and/or claimed herein, and/or further comprising one or more steps and/or functionality of any one and/or another of the method embodiments disclosed herein.

These and other embodiments will become even clearer with reference to the detailed description and figures, a brief description of which is provided immediately below.

### BRIEF DESCRIPTION OF THE DRAWINGS

The patent or application file contains at least one drawing executed in color. Copies of this patent or patent application publication with color drawings will be provided by Office upon request and payment of the necessary fee. The disclosure can be more fully understood by reading the following detailed description of the embodiment, with reference made to the accompanying drawings as follows.

FIGS. 1A-9C comprise illustrations of various embodiments of the present disclosure for Modular & Functional Connector Aspects:

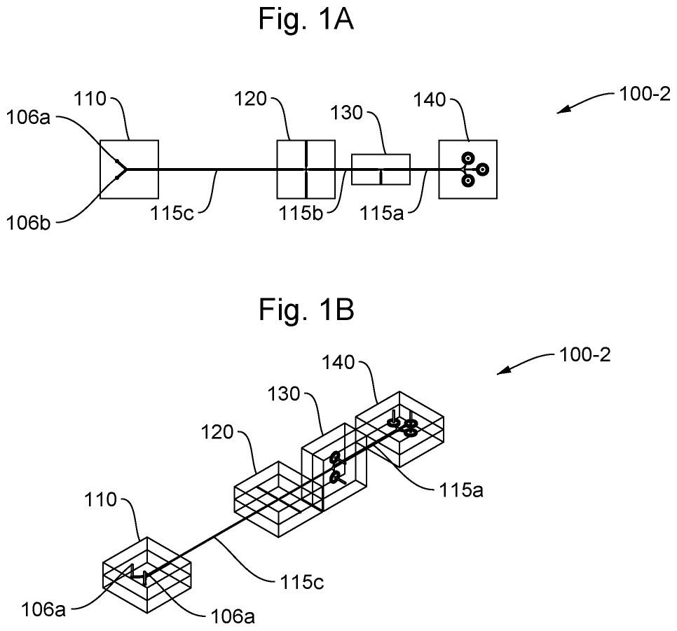

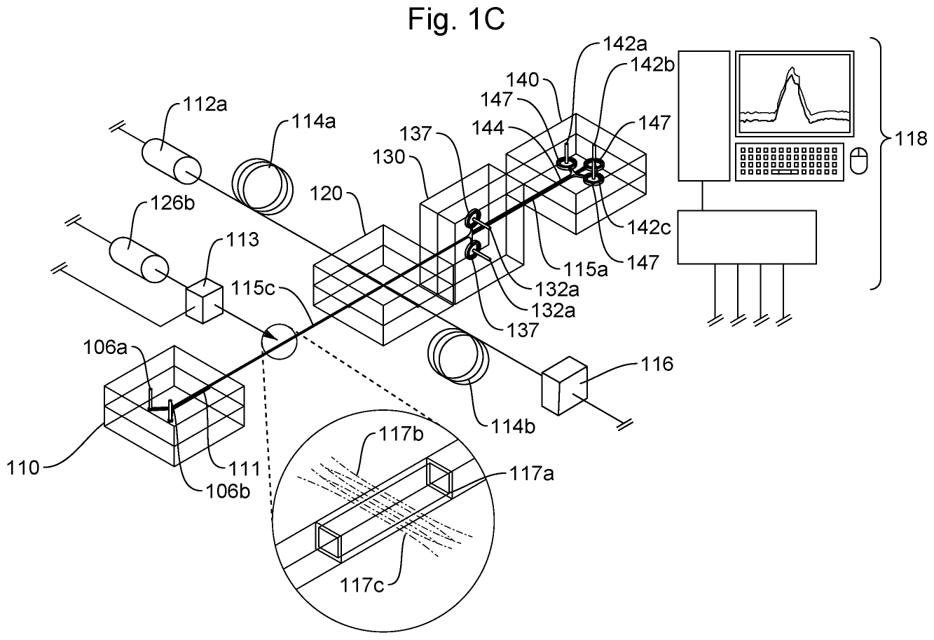

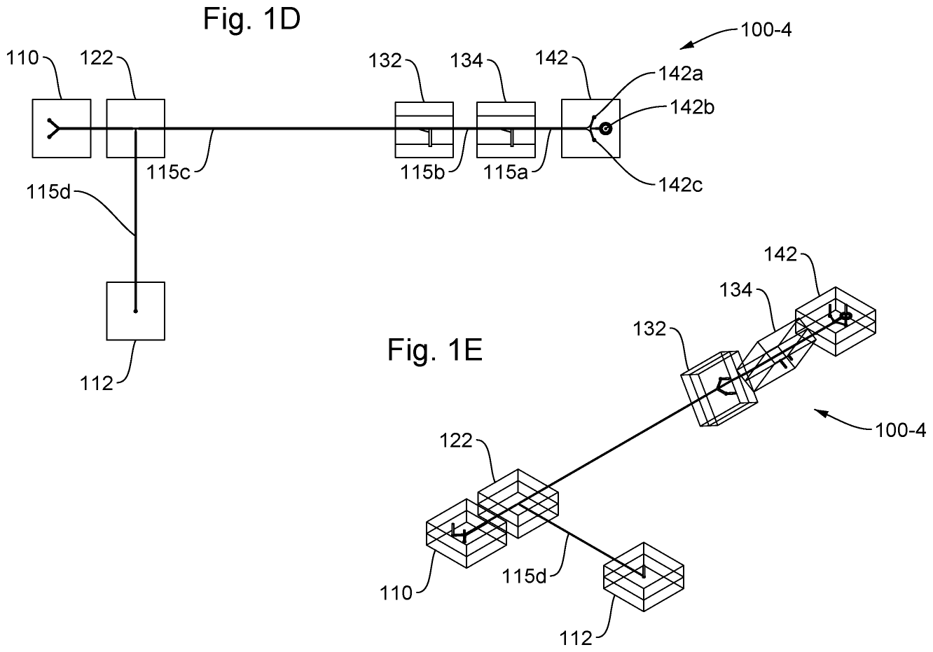

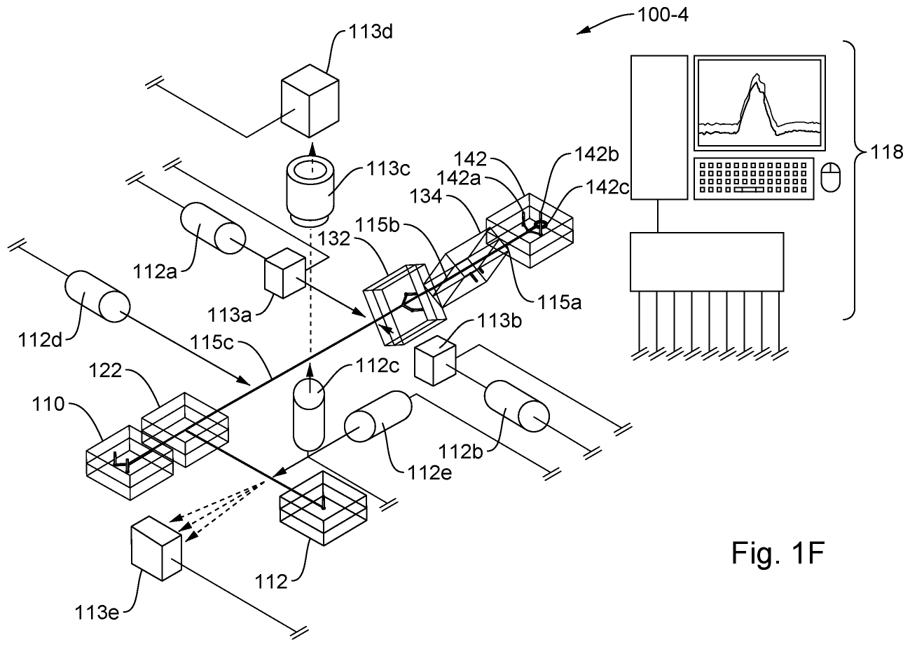

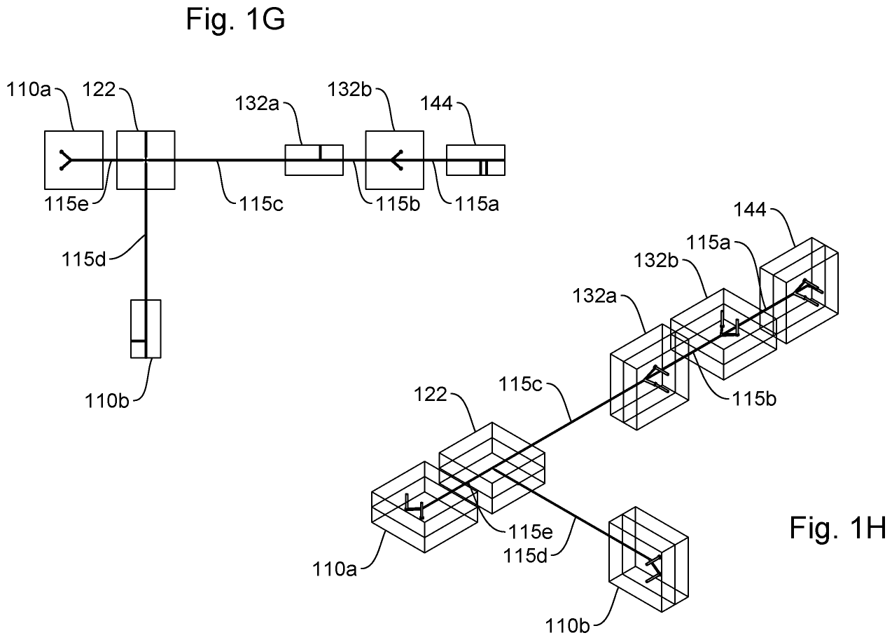

a. FIGS. 1A-1C are illustrations of a particular arrangement for a four (4) module microfluidic system according to some embodiments of the present disclosure, where FIG. 1A illustrates a top view, FIG. 1B illustrates a perspective view thereof, and FIG. 1C illustrates a particular arrangement of functionality for one functional connector for the four-module system, according to some embodiments;

b. FIGS. 1D-1F are illustrations of a particular arrangement for a six (6) module microfluidic system according to some embodiments of the present disclosure, where FIG. 1D illustrates a top view, FIG. 1E illustrates a perspective view thereof, and FIG. 1F illustrates a particular arrangement of functionality for two (2) functional connectors for the six-module system, according to some embodiments;

c. FIGS. 1G-1I are illustrations of a particular arrangement for another six (6) module microfluidic system according to some embodiments of the present disclosure, where FIG. 1G illustrates a top view, FIG. 1H illustrates a perspective view thereof, and FIG. 1I illustrates a particular arrangement of functionality for two functional connectors for the six-module system, according to some embodiments;

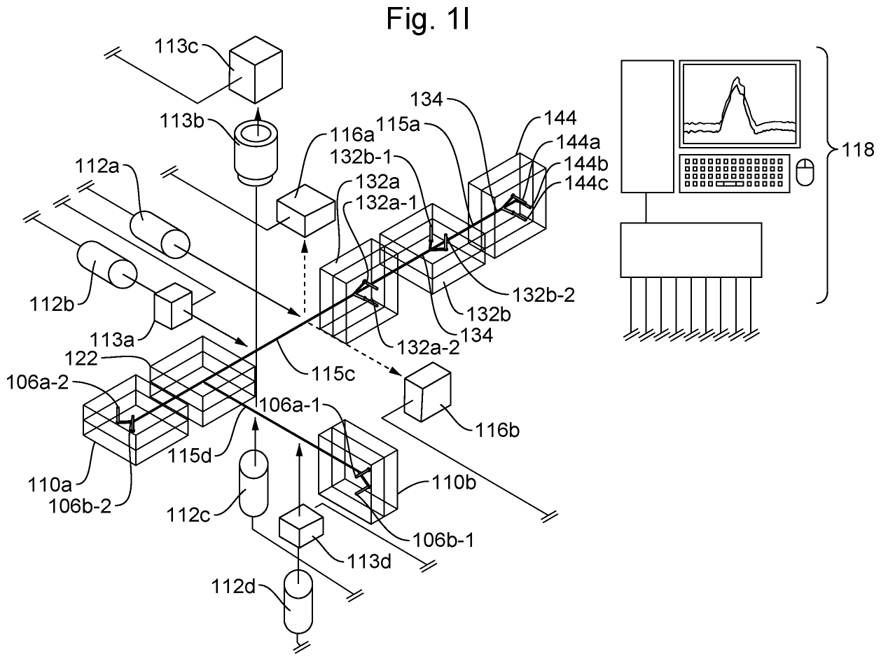

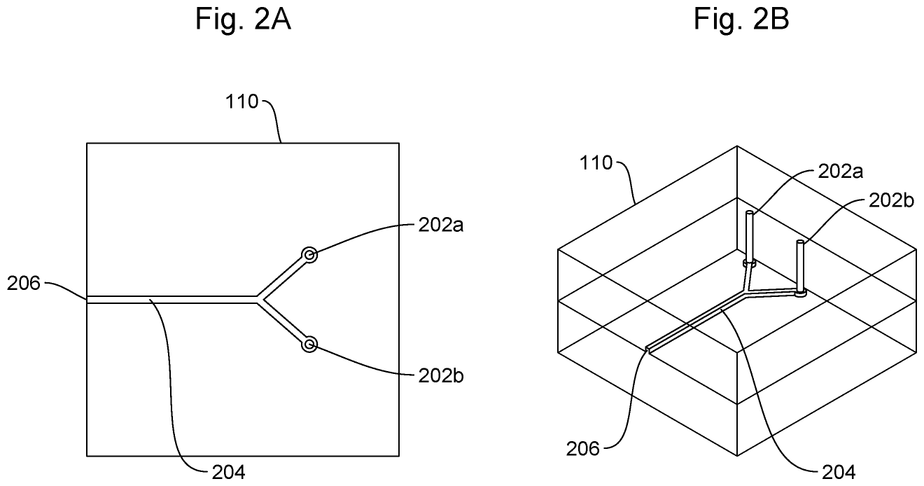

d. FIGS. 2A-2B are illustrations of one of the modules for a modular microfluidic system according to some embodiments of the present disclosure, where FIG. 2A illustrates a top view and FIG. 2B illustrates a perspective view thereof;

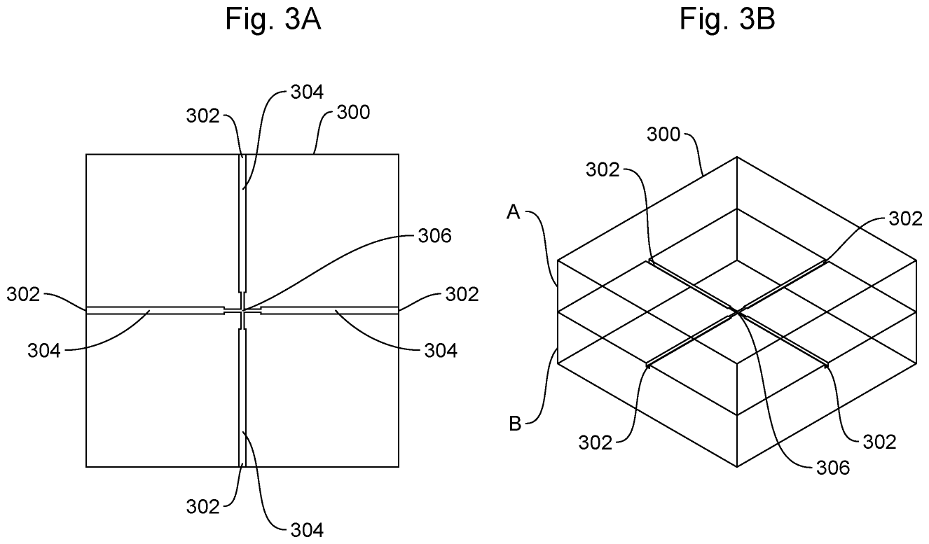

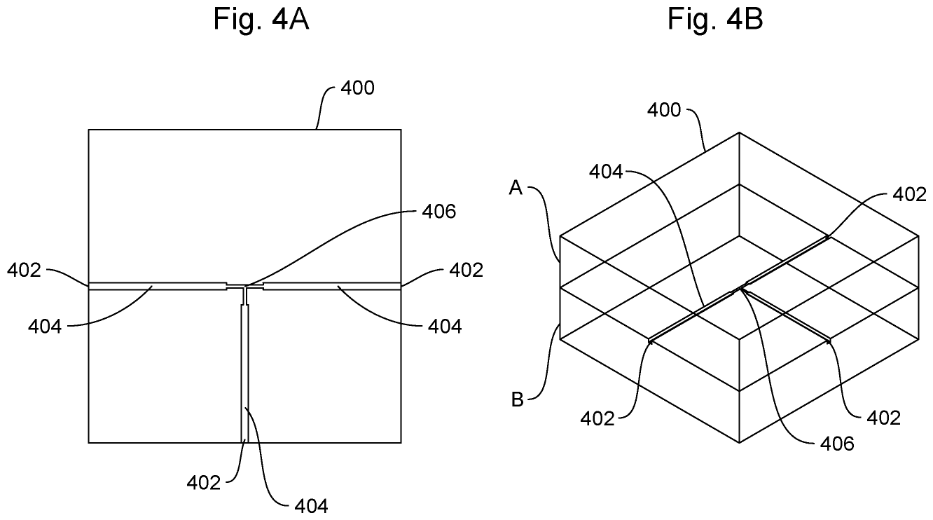

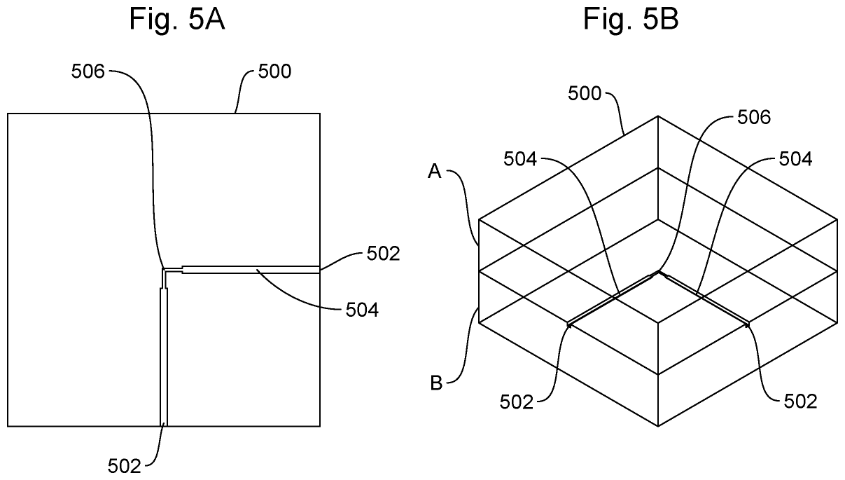

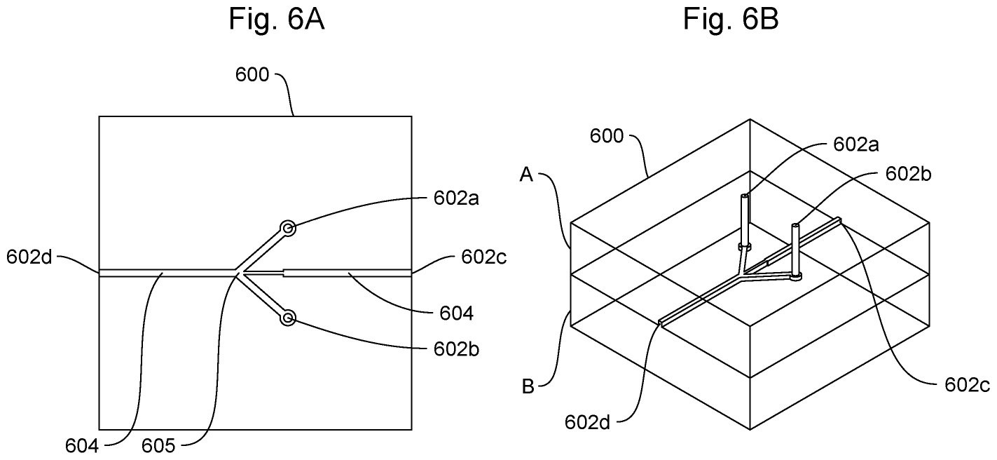

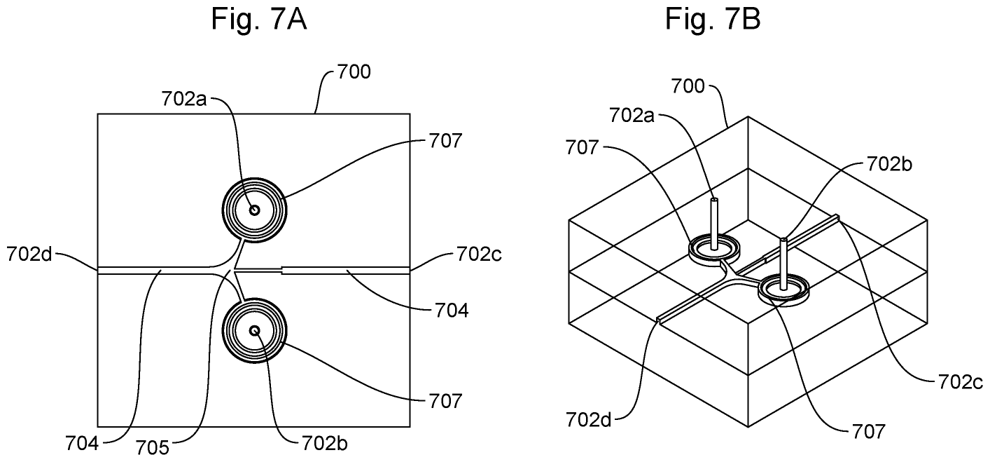

e. FIGS. 3A-3B are illustrations of one of the modules for a modular microfluidic system according to some embodiments of the present disclosure, where FIG. 3A illustrates a top view and FIG. 3B illustrates a perspective view thereof;

f. FIGS. 4A-4B are illustrations of one of the modules for a modular microfluidic system according to some embodiments of the present disclosure, where FIG. 4A illustrates a top view and FIG. 4B illustrates a perspective view thereof;

g. FIGS. 5A-5B are illustrations of one of the modules for a modular microfluidic system according to some embodiments of the present disclosure, where FIG. 5A illustrates a top view and FIG. 5B illustrates a perspective view thereof;

h. FIGS. 6A-6B are illustrations of one of the modules for a modular microfluidic system according to some embodiments of the present disclosure, where FIG. 6A illustrates a top view and FIG. 6B illustrates a perspective view thereof;

i. FIGS. 7A-7B are illustrations of one of the modules for a modular microfluidic system according to some embodiments of the present disclosure, where FIG. 7A illustrates a top view and FIG. 7B illustrates a perspective view thereof;

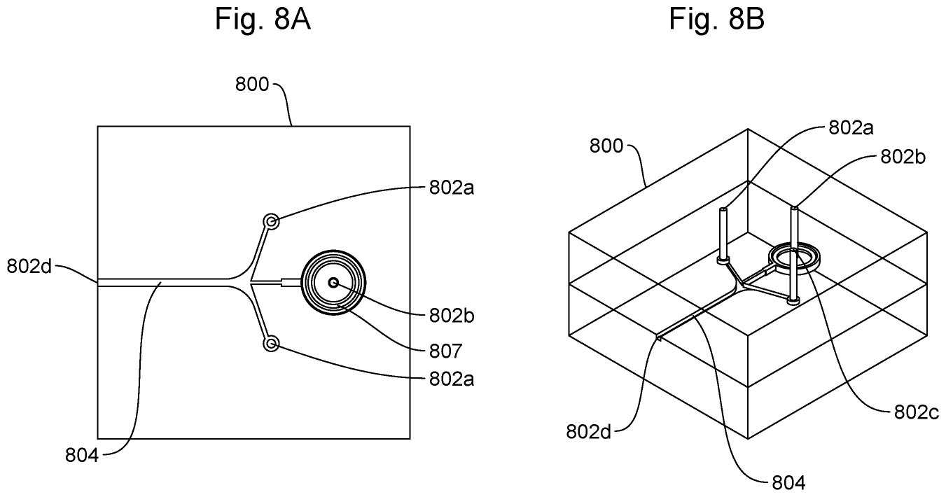

j. FIGS. 8A-8B are illustrations of one of the modules for a modular microfluidic system according to some embodiments of the present disclosure, where FIG. 8A illustrates a top view and FIG. 8B illustrates a perspective view thereof;

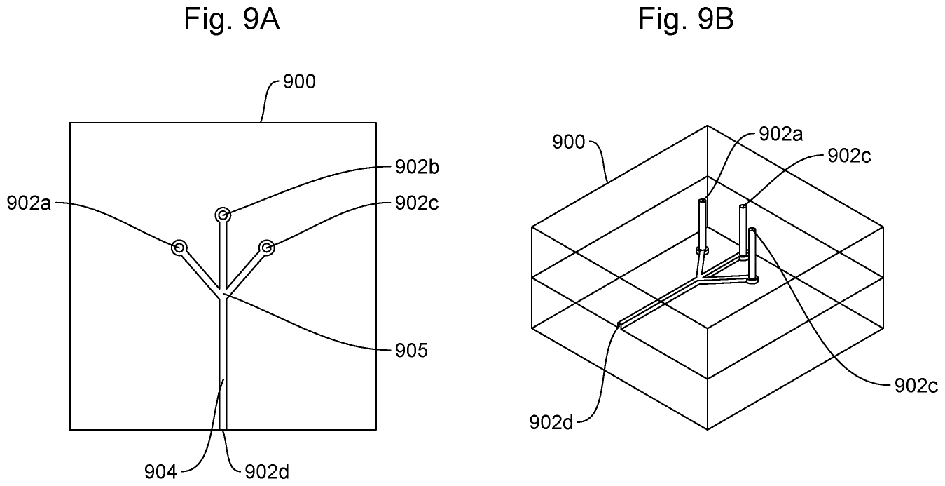

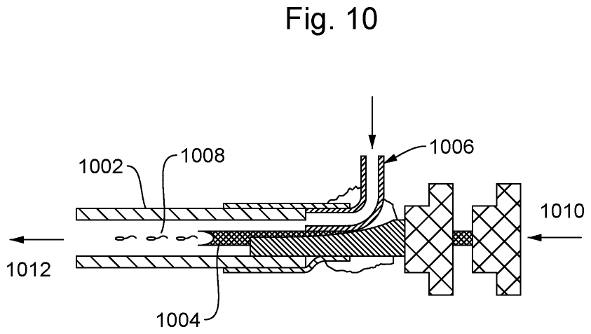

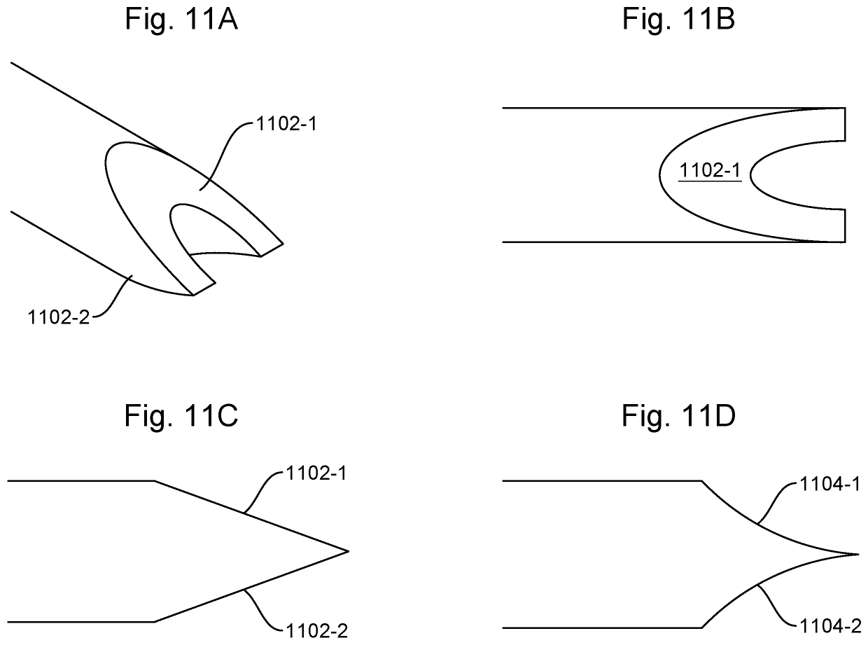

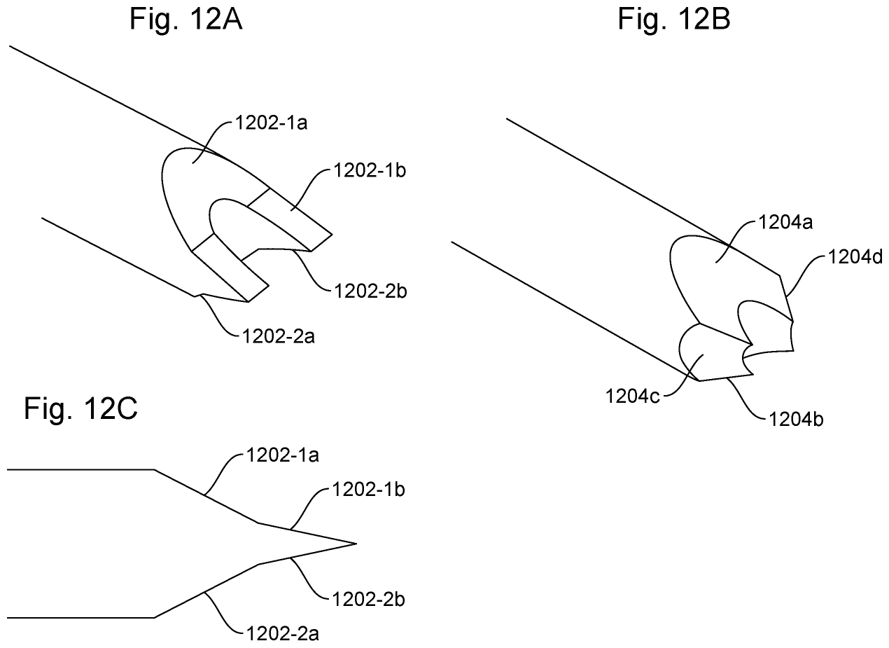

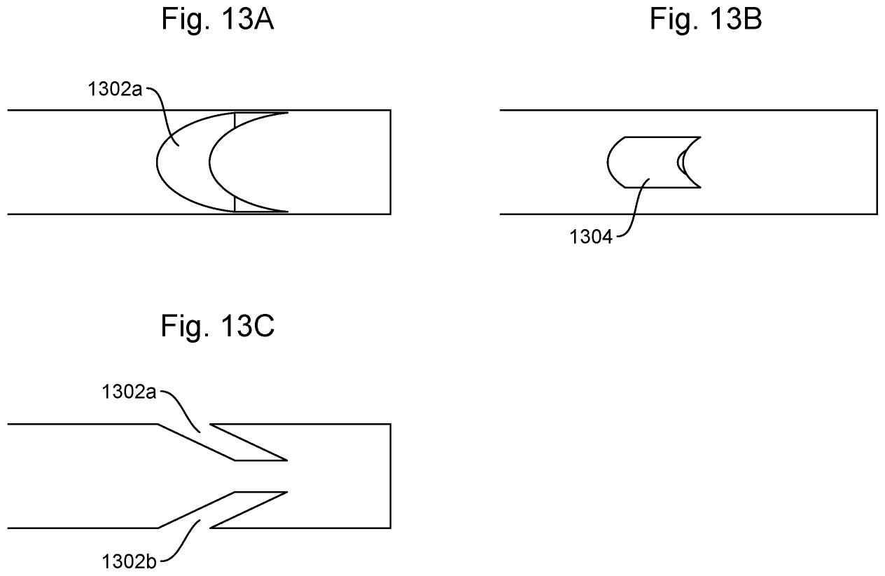

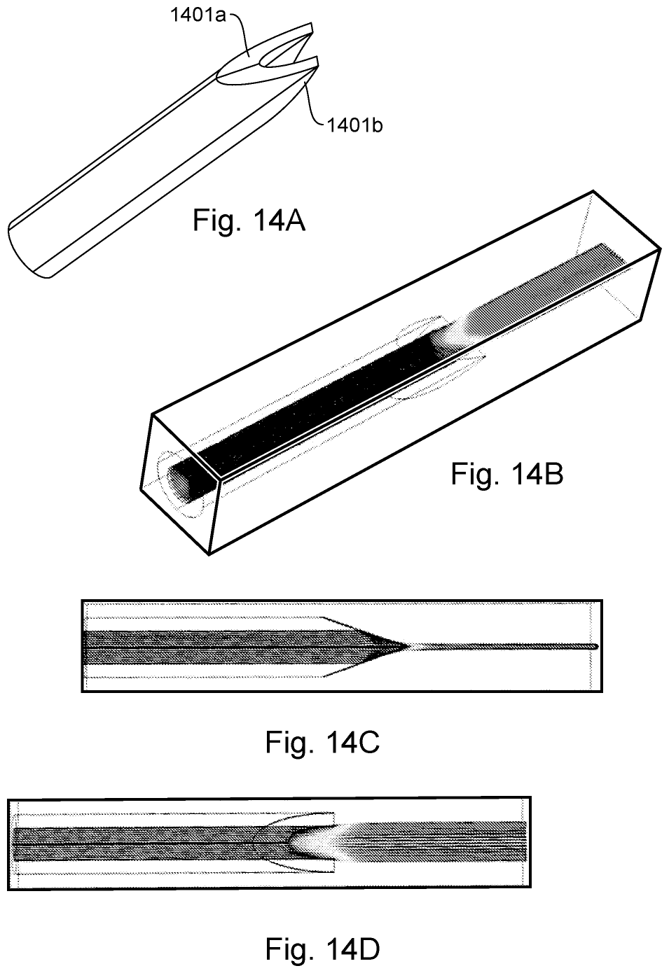

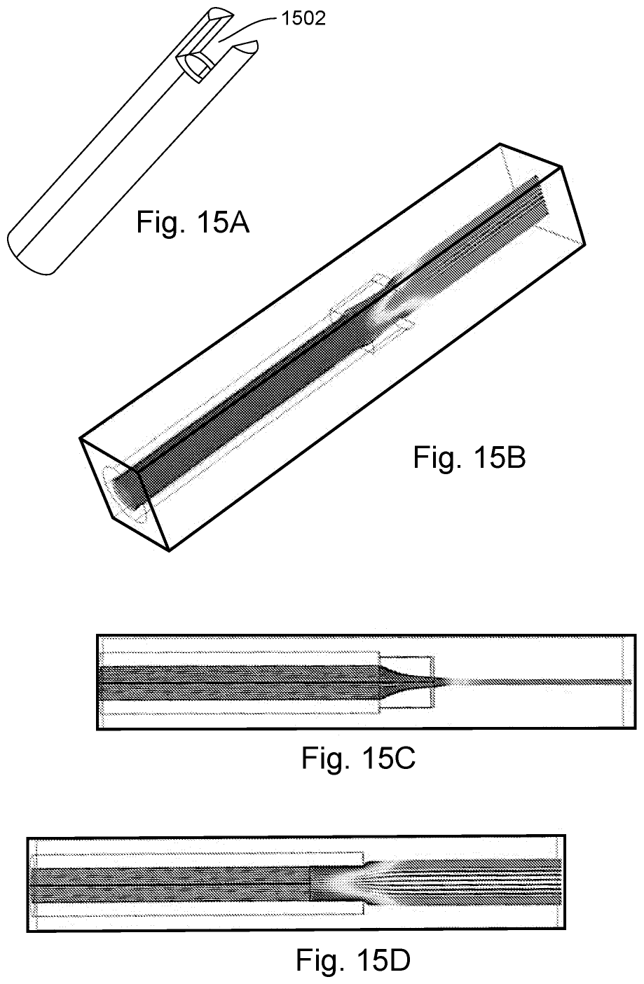

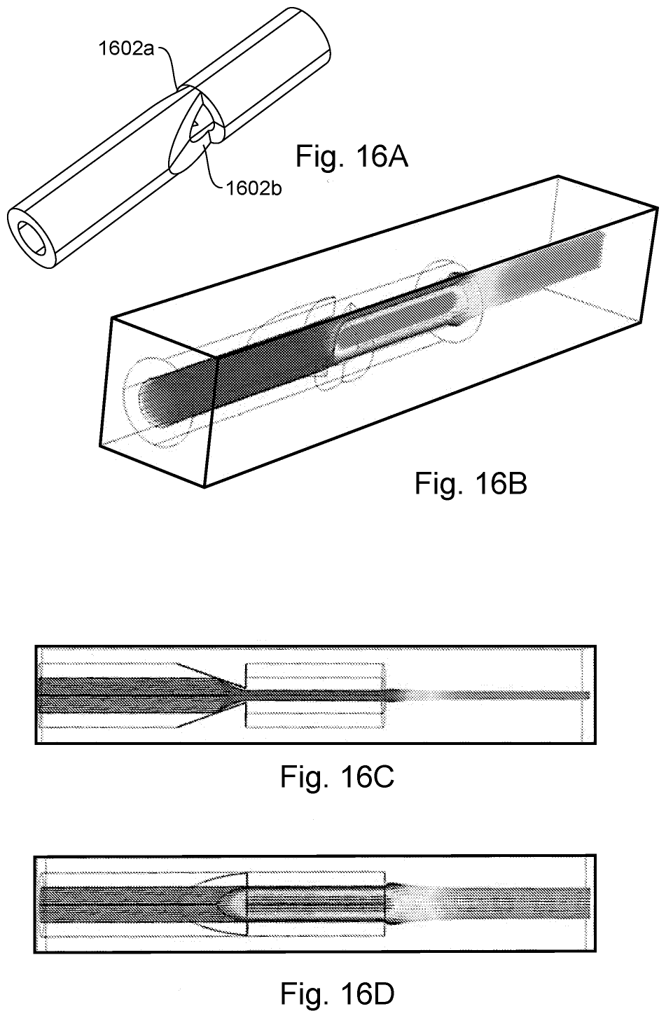

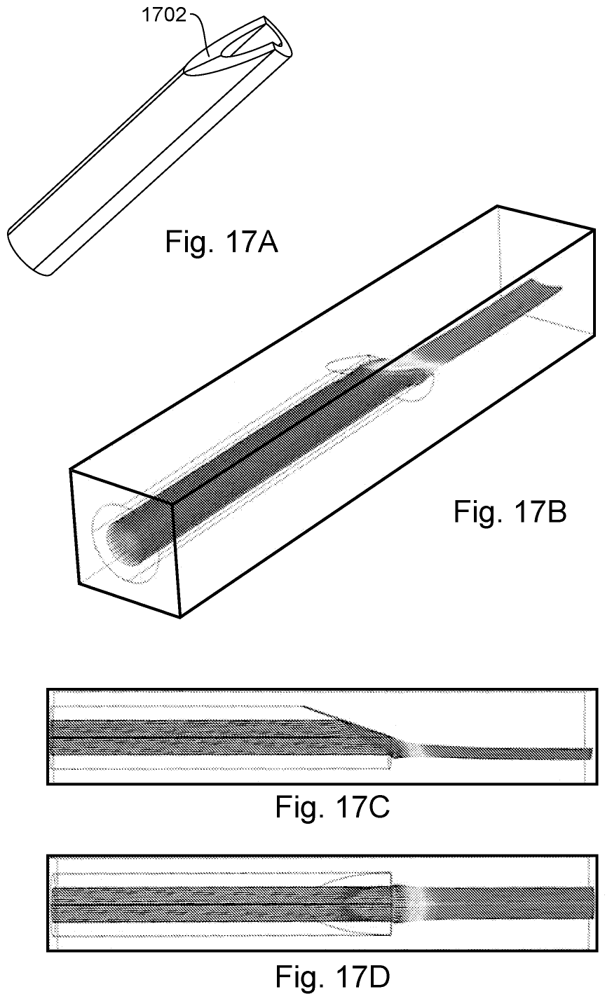

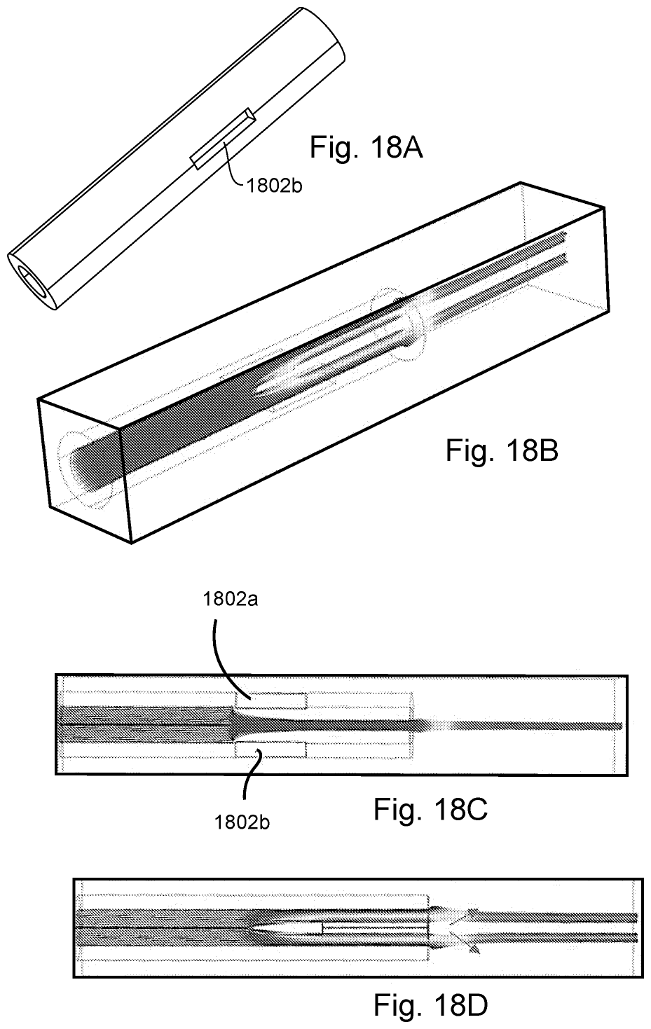

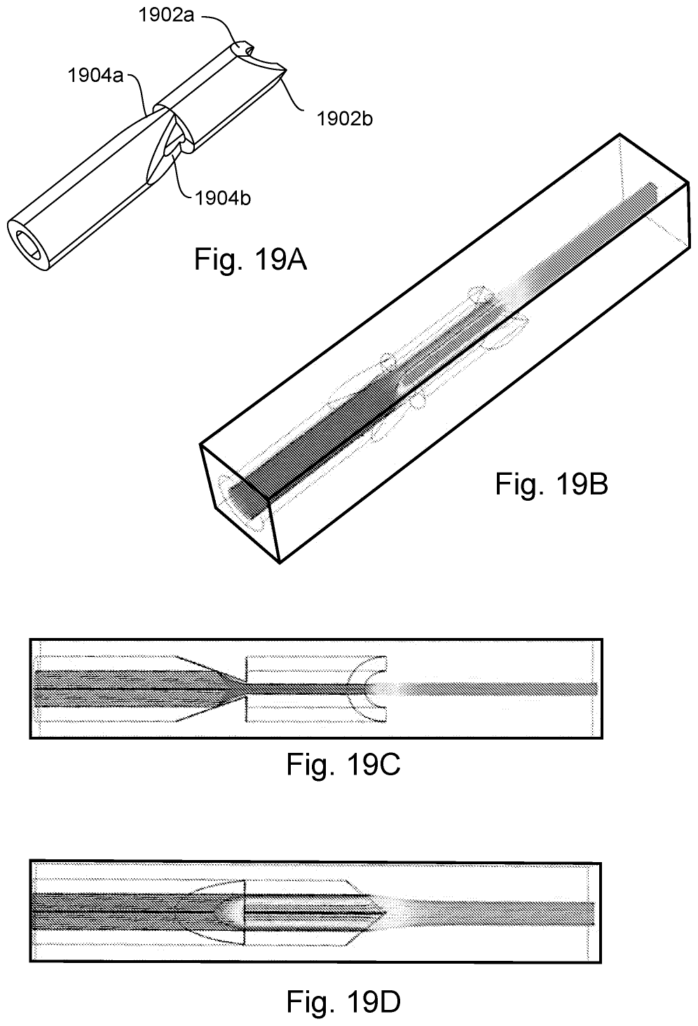

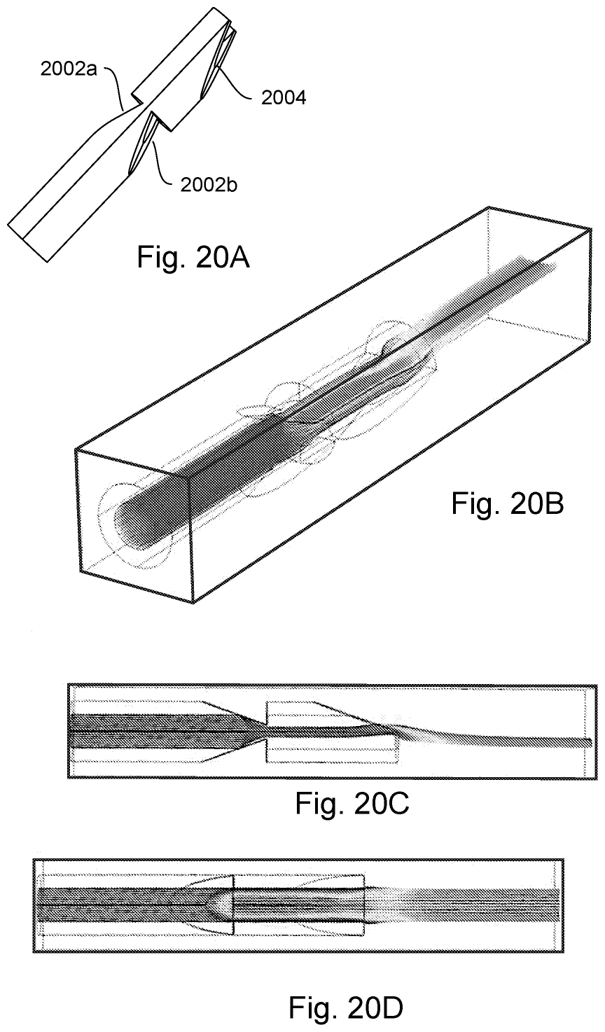

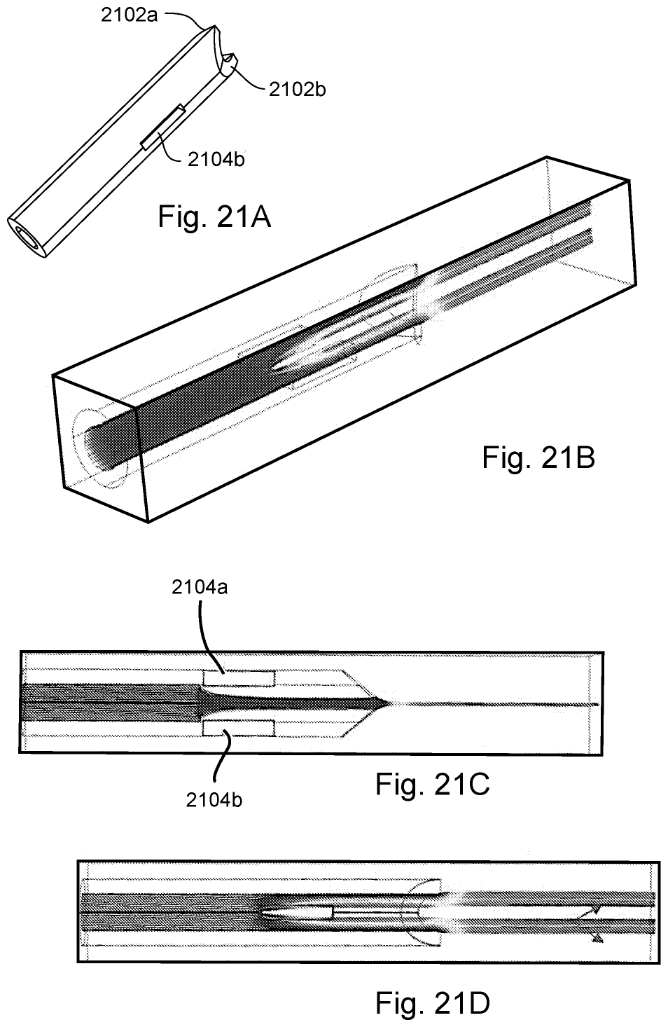

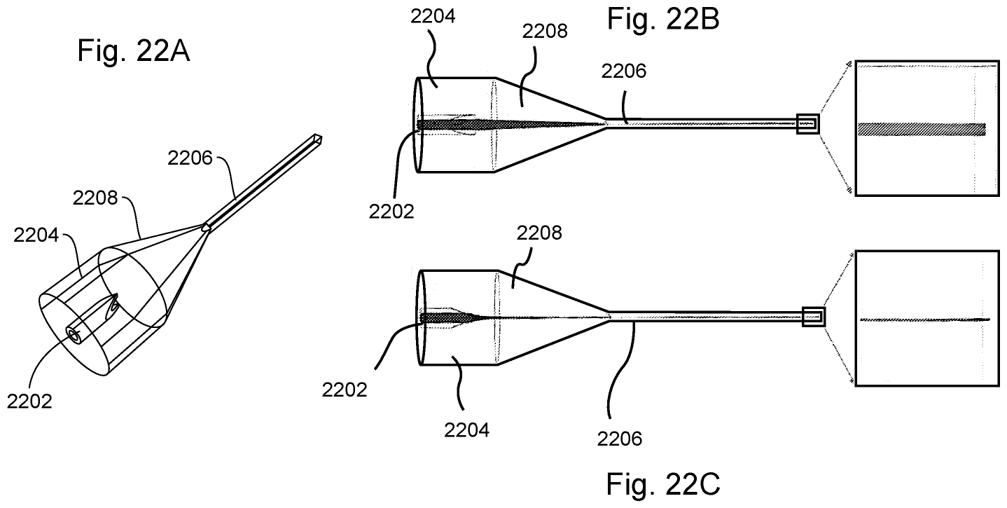

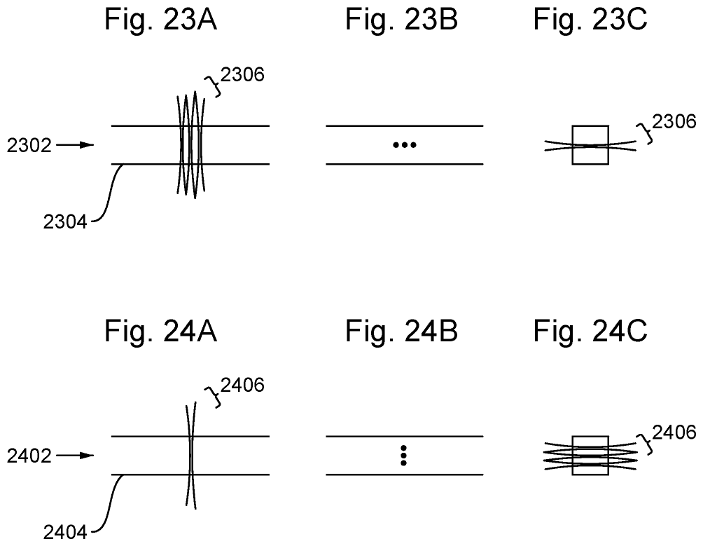

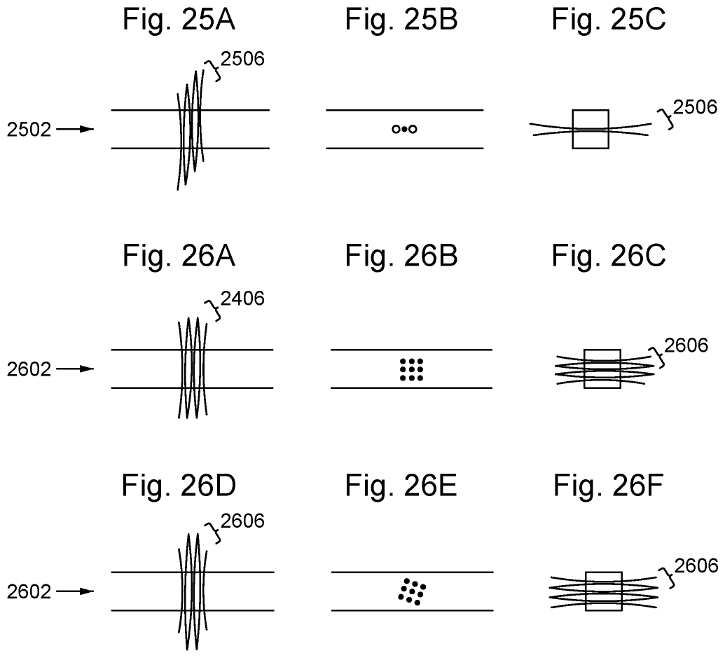

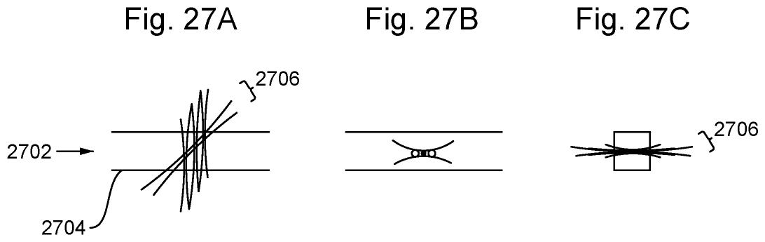

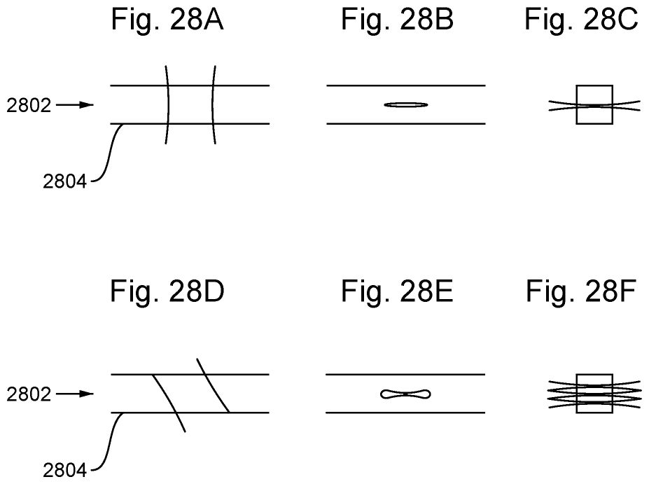

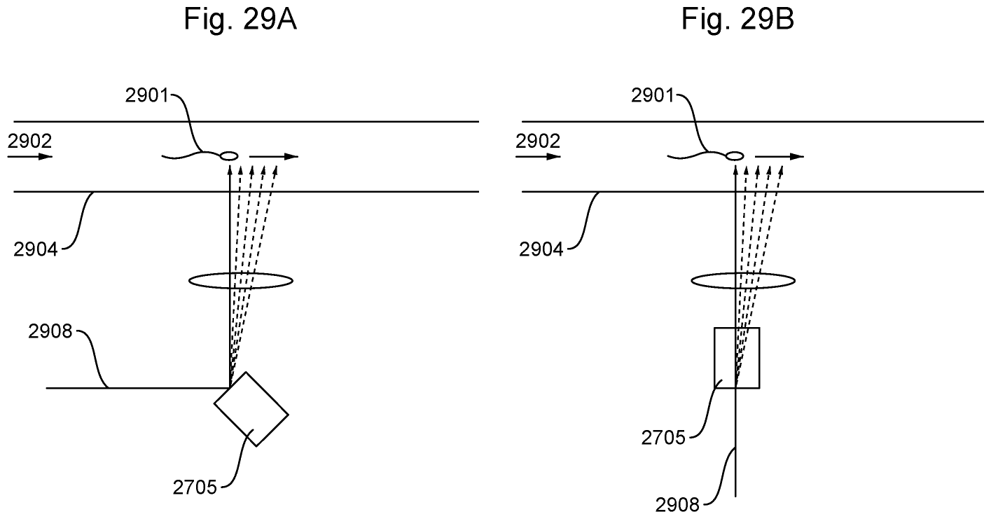

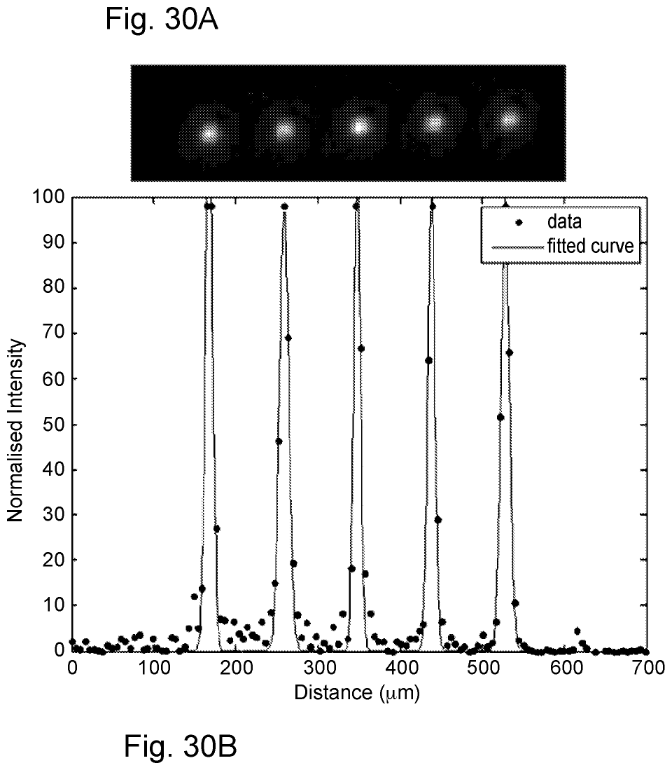

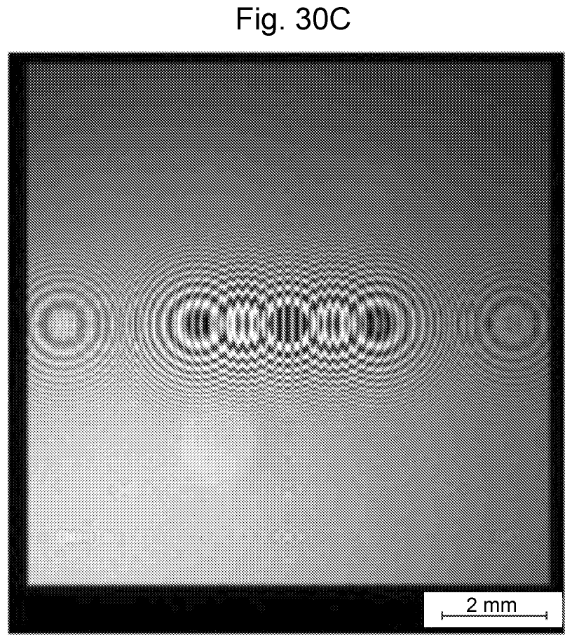

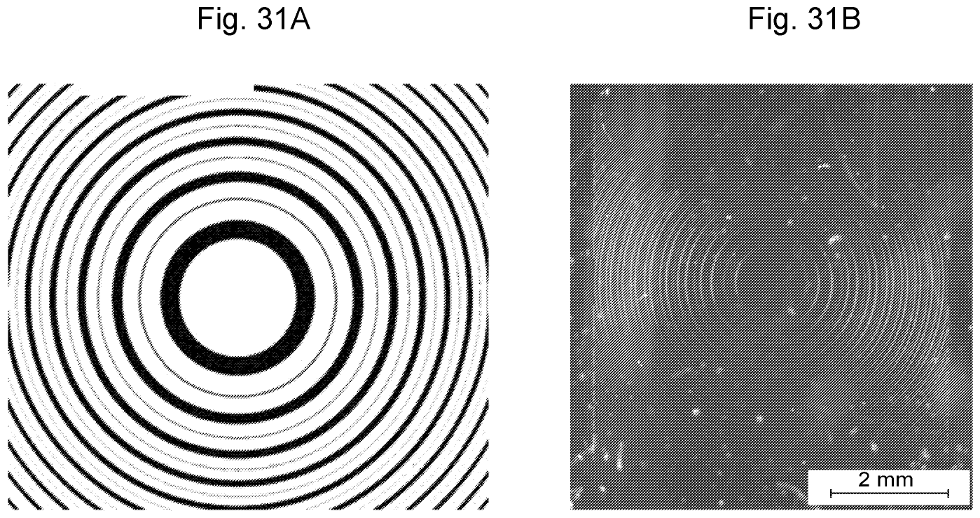

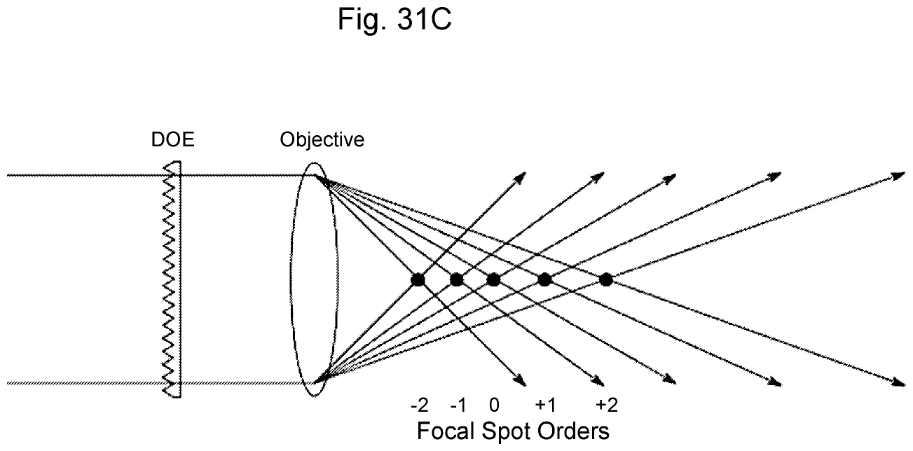

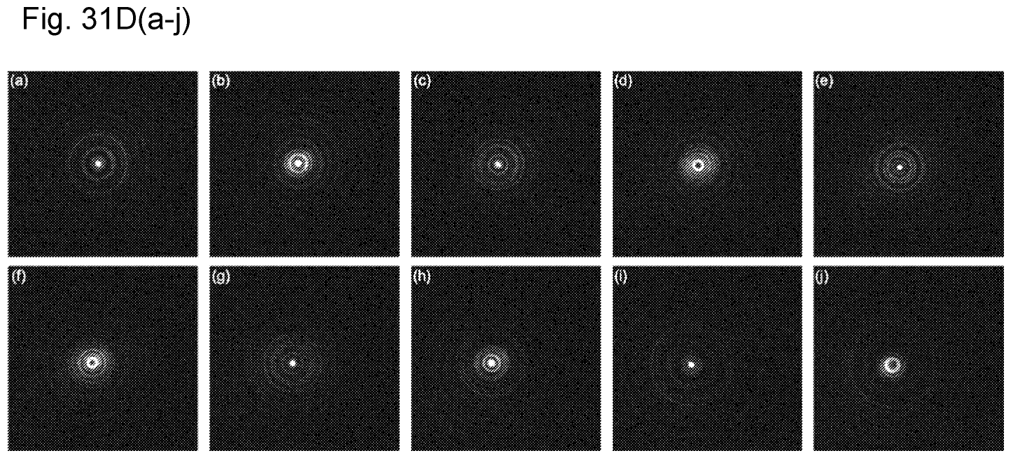

k. FIGS. 9A-9B are illustrations of one of the modules for a modular microfluidic system according to some embodiments of the present disclosure, where FIG. 9A illustrates a top view, and FIG. 9B illustrates a perspective view thereof, according to some embodiments of the present disclosure.

FIGS. 10-22C comprise illustrations of various embodiments of the present disclosure for Particle Orientation & Delivery Tube Aspects (PODT):

a. FIG. 10 illustrates a side, cross-sectional view with cutaways for clarity of particle orientation component, stage or module for a particle sorting system, according to some embodiments of the present disclosure;

b. FIGS. 11A-11D illustrate various views of a PODT, in particular, views of a distal end thereof, which includes a first type/set of structural feature(s) for imparting an orientation of particles contained in a fluid flow exiting the distal end:

FIG. 11A is a perspective view of the distal end of a PODT illustrating a first feature/set of features for imparting an orientation of particles;

FIG. 11B is a side view of the distal end of a PODT of FIG. 11A;

FIG. 11C is a top view of the distal end of the orientation tube of FIG. 11A; and

FIG. 11D is a side view of the distal end of a PODT, which while similar to those of FIGS. 11A-11C, the illustrated embodiment includes a curved chamfer instead of a straight chamfer;

c. FIGS. 12A-12C illustrate various view of a PODT, in particular, views of a distal end thereof, which includes a second type/set of structural feature(s) for imparting an orientation of particles contained in a fluid flow exiting the distal end:

FIG. 12A is a perspective view of the distal end of PODT illustrating the second feature/set of feature(s) for imparting an orientation of particles;

FIG. 112B is a side view of the distal end of the PODT of FIG. 112A; and

FIG. 12C is a perspective view of a PODT, which while similar to those of FIGS. 12A-12B, the illustrated embodiment includes multiple chamfer angles on the top and sides of the PODT instead of multiple chamfer angles on the sides alone;

d. FIG. 13A-13C illustrate various views of a PODT, in particular, views of a distal end thereof, which includes a third feature/set of feature(s) for imparting an orientation of particles contained in a fluid flow exiting the distal end:

FIG. 13A is a top view of the distal end of the PODT illustrating the third feature/set of feature(s) for imparting an orientation of particles;

FIG. 13B is a side view of the PODT of FIG. 13A;

FIG. 13C is a top view of a PODT, which while similar to those of FIGS. 13A-13B, the illustrated embodiment includes a feature that is less wide than the outer diameter of the PODT instead of equal to the outer diameter;

e. FIG. 14A illustrates a perspective view of the distal end of a PODT corresponding to the feature/set of feature(s) in FIGS. 11A-11C;

f. FIGS. 14B-14D are illustrations of flow simulations (color image) of fluid flow through the PODT of FIG. 14A;

FIG. 14B is a perspective view of the flow simulation;

FIG. 14C is a side view of the flow simulation (e.g., see FIG. 11B);

FIG. 14D is a top view of the flow simulation (e.g., see FIG. 11C);

g. FIG. 15A illustrates a perspective view of the distal end of a PODT which includes a fourth feature/set of feature(s) for imparting an orientation of particles contained in a fluid flow exiting the distal end;

h. FIGS. 15B-15D are illustrations of flow simulations (color image) of the PODT of FIG. 15A;

FIG. 15B is a perspective view of the flow simulation;

FIG. 15C is a side view of the flow simulation;

FIG. 15D is a top view of the flow simulation;

i. FIG. 16A illustrates a perspective view of the distal end of an PODT corresponding to an example within the feature/set of feature(s) shown in FIGS. 13A-13C;

j. FIGS. 16B-16D are illustrations of flow simulations (color image) of the PODT of FIG. 16A;

FIG. 16B is a perspective view of the flow simulation;

FIG. 16C is a side view of the flow simulation;

FIG. 16D is a top view of the flow simulation;

k. FIG. 17A illustrates a perspective view of the distal end of a PODT corresponding to a single chamfer;

l. FIGS. 17B-17D are illustrations of flow simulations (color image) of the PODT of FIG. 17A;

FIG. 17B is a perspective view of the flow simulation;

FIG. 17C is a side view of the flow simulation;

FIG. 17D is a top view of the flow simulation;

m. FIG. 18A illustrates a perspective view of the distal end of a PODT corresponding to an example within the feature/set of feature(s) shown in FIGS. 13A-13C and FIG. 16A;

n. FIGS. 18B-18D are illustrations of flow simulations (color image) of the PODT of FIG. 18A;

FIG. 18B is a perspective view of the flow simulation;

FIG. 18C is a side view of the flow simulation;

FIG. 18D is a top view of the flow simulation;

o. FIG. 19A illustrates a perspective view of the distal end of a PODT corresponding to a combination feature set, including a notched portion prior to a chamfered portion;

p. FIGS. 19B-19D are illustrations of flow simulations (color image) of the orientation tube of FIG. 19A;

FIG. 19B is a perspective view of the flow simulation;

FIG. 19C is a side view of the flow simulation;

FIG. 19D is a top view of the flow simulation;

q. FIG. 20A illustrates a perspective view of the distal end of a PODT corresponding to a combination feature set, similar to that of FIG. 19A, including a notched portion prior to a chamfered portion, but with the chamfered portion being rotated 90 degrees relative to the notch positions and with a single chamfer rather than two opposite chamfers;

r. FIGS. 20B-20D are illustrations of flow simulations (color image) of the PODT of FIG. 20A;

FIG. 20B is a perspective view of the flow simulation;

FIG. 20C is a side view of the flow simulation;

FIG. 20D is a top view of the flow simulation;

s. FIG. 21A illustrates a perspective view of the distal end of a PODT corresponding to a combination feature set, combining the features illustrated in FIG. 18A, with those from FIG. 11A;

t. FIGS. 21B-21D are illustrations of flow simulations (color image) of the PODT of FIG. 21A;

FIG. 21B is a perspective view of the flow simulation;

FIG. 21C is a side view of the flow simulation;

FIG. 21D is a top view of the flow simulation;

u. FIG. 22A illustrates a perspective view of the distal end of a PODT, similar to that illustrated in FIG. 10A, as well as a chamber and microfluidic tube which follows thereafter;

and

v. FIGS. 22B-21C are illustrations of flow simulations (color image) of the orientation tube/chamber of FIG. 22A

FIGS. 23-32 comprise illustrations of various embodiments of the present disclosure for Spatial Patterning Aspects:

a. FIGS. 23A-23C illustrate one (1) example of a simple static or dynamic pattern generated by shaping a single RS into multiple radiation beams parallel and separated along the axis of fluid flow in the microfluidic channel or functional connector, according to some embodiments of the disclosure;

FIG. 23A is a top view of the microfluidic channel or functional connector;

FIG. 23B is a side view of the microfluidic channel or functional connector;

FIG. 23C is the viewpoint along the direction of fluid flow in a microfluidic channel or functional connector;

b. FIGS. 24A-24C illustrate a second example of a simple static or dynamic pattern generated by shaping a single RS into multiple radiation beams parallel and separated perpendicular to the axis of fluid flow in the microfluidic channel or functional connector, according to some embodiments of the disclosure;

FIG. 24A is a top view of the microfluidic channel or functional connector;

FIG. 24B is a side view of the microfluidic channel or functional connector;

FIG. 24C is the viewpoint along the direction of fluid flow in a microfluidic channel or functional connector;

c. FIGS. 25A-25C illustrate a third example of a simple static or dynamic pattern generated by shaping a single RS into multiple radiation beams parallel and separated along the axis of fluid flow in the microfluidic channel or functional connector, according to some embodiments of the disclosure, which is similar to FIGS. 23A-23C except that the focal points of the multiple beams are at different points along the direction of propagation of the radiation and perpendicular to the axis of fluid flow in the microfluidic channel or functional connector;

FIG. 25A is a top view of the microfluidic channel or functional connector;

FIG. 25B is a side view of the microfluidic channel or functional connector;

FIG. 25C is the viewpoint along the direction of fluid flow in a microfluidic channel or functional connector;

d. FIGS. 26A-26F illustrate two (2) examples of a more complex static or dynamic pattern generated by shaping a single RS into multiple radiation beams in a two-dimensional array in a plane that includes the axis of fluid flow in in the microfluidic channel or functional connector, according to some embodiments of the disclosure;

FIG. 26A and FIG. 26D are top views of the microfluidic channel or functional connector;

FIG. 26B and FIG. 26E are a side views of the microfluidic channel or functional connector;

FIG. 26C and FIG. 26F are the viewpoint along the direction of fluid flow in a microfluidic channel or functional connector;

e. FIGS. 27A-27C illustrate one (1) example of a more complex static or dynamic pattern generated by shaping more than one RS into multiple radiation beams along multiple radiation propagation axes relative to the axis of fluid flow in the microfluidic channel or functional connector, according to some embodiments of the disclosure;

FIG. 27A is a top view of the microfluidic channel or functional connector;

FIG. 27B is a side view of the microfluidic channel or functional connector;

FIG. 27C is the viewpoint along the direction of fluid flow in a microfluidic channel or functional connector;

f. FIGS. 28A-28F illustrate two (2) example of more complex static or dynamic patterns generated by shaping more than one RS into a line focused beam along with the line focus aligned completely (FIG. 28A-28C) or partially (FIG. 28D-28F) along the axis of fluid flow in the microfluidic channel or functional connector, according to some embodiments of the disclosure;

FIG. 28A and FIG. 28D are top views of the microfluidic channel or functional connector;

FIG. 28B and FIG. 28E are side views of the microfluidic channel or functional connector;

FIG. 28C and FIG. 28F are viewpoints along the direction of fluid flow in a microfluidic channel or functional connector;

g. FIG. 29A-29B illustrates two examples of controlled dynamical patterns, the particular embodiment of which is the controlled movement of the radiation beam(s) along the axis of fluid flow in the microfluidic channel or functional connector, according to some embodiments of the disclosure; the diagrams are top views of the microfluidic channel or functional connector;

h. FIG. 30A-30C illustrate an example similar to the pattern observed in FIGS. 23A-23C, the particular difference between FIG. 30 and FIG. 23 being that there are five (5) separate radiation beams rather than three (3) radiation beams parallel and separated along the axis of fluid flow in the microfluidic channel or functional connector;

FIG. 30A is an image of the five focal spots in the same plane parallel to the axis of fluid flow in the microfluidic channel or functional connector, as in the view shown in FIG. 23B;

FIG. 30B is an intensity measurement as a function of distance along the axis of fluid flow in the microfluidic channel or functional connector, providing quantification of the brightness of the focal spots in FIG. 30A;

FIG. 30C is a microscope image of the diffractive optical element that was used to create the pattern of the five parallel radiation beams shown in FIG. 30A and FIG. 30B;

i. FIG. 31A-30D illustrate an example similar to the pattern observed in FIGS. 25A-25C, the particular differences between FIG. 31 and FIG. 25 being that there are five (5) separate radiation beams rather than three (3) radiation beams focused at different focal points along the direction of propagation of the radiation and perpendicular to the axis of fluid flow in the microfluidic channel or functional connector, and that the multiple beams in FIG. 31 are not separated along the axis defined by the fluid flow;

FIG. 31A is a computer generated diffractive optical element pattern needed to generate the five cascading focal spots in FIG. 31C and FIG. 31D;

FIG. 31B is a microscope image of the diffractive optical element used to generate the five cascading focal spots in FIG. 31C and FIGS. 31D-M;

FIG. 31C is a diagram of the pattern of five cascading focal spots generated by the diffractive optical element in FIG. 31B and shown in FIG. 31D;

At least some of the various embodiments of corresponding inventions and associated combinations thereof will be even clearer with reference to the following detailed description.

### DETAILED DESCRIPTION OF AT LEAST SOME EMBODIMENTS

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although systems, devices, structure, functionality, methods, and steps, similar or equivalent to those described herein can be used in the practice or testing of the present disclosure, suitable systems, devices, structure, functionality, methods, and steps, are described below. All publications, patent applications, patents, and other references mentioned herein are incorporated by reference in their entireties for all purposes. The references cited herein are not admitted to be prior art to the claimed disclosure. In the case of conflict, the present specification, including definitions, will control. In addition, the materials, methods, and examples are illustrative only and are not intended to be limiting.

### Modular & Functional Connector Aspects

As shown in FIGS. 1-9C, a modular, microfluidic sorting system for sorting particles in a microfluidic system is provided. In FIGS. 1A-C, illustrate four (4) module 100-2, and FIGS. 1D-1I illustrate various six (6) module 100-4, 100-6 systems, each module including at least one designated function and connected to at least one other module (e.g., an adjacent module) via a functional connector. In some embodiments, modules are configured to be arranged in a plurality of configurations, depending upon at least one of the number and type of modules provided, the one or more functions carried out by each module, the functions of the connectors therebetween, and the desired overall functionality of the system as a whole. The disclosed systems of these figures, generally, operate to at least one, in a particle flow, illuminating particles, identifying particles, imaging particles, orientating particles, and sorting particles. One or more connectors provided between modules, in some embodiments, are configured to, in addition to flowing fluid/particles between modules, to perform at least one additional functions, which can include, for example, any of illuminating, imaging, orientating, and sorting particles. Each module can be made, for example, in “halves,” e.g., half A and half B (see FIGS. 3A-3B)—such that structure is machined/etched out of each half, then the two halves are assembled together via, for example, welding, or an adhesive. However, one of skill in the art appreciates that there are other ways to make the modules.

Each module within the modular microfluidic system need not be fixed relative to each other, indeed, they may be oriented in any manner. For example, as shown in the figures, certain modules can be rotated relative to other modules (and/or the system as a whole), and, in some embodiments, rotated orthogonal to one or more other modules. See e.g., modules 132, 134, and 110, the latter in FIG. 1I.

FIG. 1C illustrates a modular sorting system, according to some embodiments, with four (4) modules and multiple connectors, at least one of which in this figure, 115c, corresponds to a functional connector (i.e., a connector which does more than just flow fluid/particles from one module to another or provide a structural link). Accordingly, particles enter the system in module 140 in the central entry port 142b and pass through a filter 147 before entering a microfluidic channel 144 (main channel). Two sheath flows enter module 140 in port 142a and 142c, each incorporating a filter 147 before the sheath fluids enter channel 144, and at least one (preferably both) of hydrodynamically position and orient the particles in the vertical plane (i.e., one plane/dimension). The particles pass through connector 115a to a second module 130. Two sheath flows enter the second module 130 through ports 132a, b positioned to each side (e.g., above and below) of channel 134, each incorporating a filter 137 before the sheath fluids enter channel 134, and at least one of (and preferably both of) hydrodynamically position and orient the particles in the horizontal plane. Together, sheath flows that enter the system through modules 140 and 130 position the particles at a desired location within the microfluidic channel (e.g. confined to a central region of the microfluidic channel), and, in some embodiments, orient the particles in a desired orientation relative to the laboratory frame of reference (e.g., with one particle feature oriented vertically relative to the laboratory frame of reference). The oriented, positioned particles pass through a second connector 115b and enter module 120 which includes a function of discriminating the particles into two or more subsets. In this module, the particles can be illuminated by a radiation source 112a (e.g., narrow band) via a fibre optic 114a configured to induce the particles to emit fluorescence. Fluorescence is transmitted by a second fibre optic 114b to an optical detector 116. The optical signal, that indicates the fluorescence signal emitted by each particle as it passes through detection region in module 120, can be transmitted electronically to a controller/signal collection/signal processor system 118. As the fluorescence from each particle is detected, system 118 determines whether to switch that particle into a different flow stream, based upon one or more features of the fluorescence signal (e.g. intensity, band shape). If the decision is positive, that is, to switch the particle into a different flow stream, the electronic system 118 sends a signal to a radiation source 112b to induce switching to illuminate the particles after passing through a radiation beam shaping system 113, as they flow through the functional connector/linker 115c, for a time to illuminate the particles so as to generate a force perpendicular to the axis of the flow stream and displace the particle horizontally (i.e., to one side or the other in microfluidic channel of the connector 115c. The radiation beam shaping unit 113, according to some embodiments, can be configured to generate three (3) parallel radiation beams 115a-c that are displaced from one another along the axis of flow of the channel, with their focal points at the same position vertically and horizontally (e.g., 2 dimensionally) within the channel (see also, e.g., FIG. 23A-23C). The particles then pass from the functional connector/linker 115c to module 110 in parallel flow streams within the same channel 111. In such embodiments, the particles can be sorted into two populations that are collected in separate output flow streams, each leaving the microfluidic system through a different port 106a, 106b (i.e., output port) in module 110.

FIG. 1F illustrates a modular sorting system 100-4 according to some embodiments with six (6) modules and at least two (2) functional connectors (i.e., connectors which do more than just flow fluid/particles from one module to another), the system being configured to sort particles into at least three separate populations (for example). The particles enter the system in the first module 142 in central entry port 142b and pass through a filter (see 807, FIGS. 8A-8B) before entering a microfluidic channel within the module. Two (2) sheath flows enter module 142 in ports 142a, 142c and at least one of (preferably both of) hydrodynamically position and orient the particles in the vertical plane (i.e., one plane/dimension). The particles pass through a connector 115a to a second module 134 which is rotated at a predetermined angle (here, 120 degrees) around the axis of flow in the microfluidic channel relative to the first module 142. Two sheath flows enter module 134 (e.g., see module 200, FIGS. 2A-2B) through ports on this module (e.g., see ports 202a, 202b, FIGS. 2A-B) and at least one of (and preferably both of) hydrodynamically position and orient the particles diagonally relative to the axis of flow in the channel. The particles pass through a second connector 115b into module 132 (e.g., see again, module 200, FIGS. 2A-2B) which is rotated at a predetermined angle (here, 120 degrees) around the axis of flow in the microfluidic channel relative to module 134, and similar additional sheath flows are included. Together, the sheath flows that enter the system through the three modules 142, 134 and 132 position the particles at a desired location within the channel, e.g., being confined to a triangular shaped central region of the channel, and orient them in a desired orientation relative to the laboratory frame of reference (e.g., with one particle feature oriented vertically relative to the laboratory frame of reference).

The oriented, positioned particles pass into a functional connector 115c that includes a plurality of functional steps, here three (3) functional steps, in the single connector. To this end, first, the particles enter a free-space optical orientation stage I comprising two radiation sources 112a, 112b and beam shaping units 113a, 113b. The shaped, radiation sources interact with each particle to induce a torque on the particle and refine its orientation (e.g., with one particle feature oriented vertically within a narrower angle range relative to the laboratory frame of reference). The optical orientation stage I is controlled by an electronic controller/signal collection/signal processing system 118. Second, the particles then pass through a discrimination stage II that comprises a radiation source 112c and an imaging system 113c, 113d such that the radiation source illuminates each particle as it passes through the microfluidic channel so that each particle is induced to emit fluorescence which is collected by the optical system 113c and transmitted to an optical image detector 113d. The optical image is transmitted electronically to the electronic system 118. As the fluorescence image from each particle is detected, the system 118 determines whether to switch that particle into a different flow stream, based upon one or more features of the fluorescence image (e.g. intensity, intensity distribution, shape). If the determination is positive (e.g. to switch the particle into a different flow steam), the electronic system 118 sends a signal to the third stage III in this functional connector, a switching station. The switching station comprises a radiation source 112d that illuminates the particles in response to a signal from the electronic system 118 so as to induce a force perpendicular to the axis of the flow stream and so displace the particles into different flow stream. In the illustrated embodiment, the particles are sorted into three different flow streams within a channel. The particles then pass into the fourth module 122, which operates to separate out one of the flow streams and direct it through an output port through another functional connector 115d to module 123. As the particles (e.g., cells) pass through connector 115d, they are counted using a light scattering stage IV comprising a radiation source 112e and optical detector 113e that converts the scattered light intensity to an electrical signal and sends it to the electronic system 118. The particles that are not directed to module 112 continue along the main axis of flow through connector 115e (see FIGS. 1D-1E) to module 110 in parallel flow streams within the same channel. The particles in this flow “sub”-stream are sorted into two populations that can be collected in separate output flow streams, each leaving the microfluidic system through a different output port of module 110.

FIG. 1I illustrates a modular sorting system, according to some embodiments, with six (6) modules and multiple connectors, at least two of which correspond to functional connectors (i.e., connectors which do more than just flow fluid/particles from one module to another). Accordingly, particles enter the system in module 144 in the central entry port 144b and enter the main microfluidic channel YYY (main channel). Two sheath flows enter module 144 in ports 144a and 144c positioned to each side (e.g., above and below) of channel 134 and at least one (preferably both) of hydrodynamically position and orient the particles in the horizontal plane. The particles pass through connector 115a to a second module 132b. Two sheath flows enter the second module 132 through ports 132b-1, 132b-2 positioned to each side of the module channel and at least one of (and preferably both of) hydrodynamically position and orient the particles in the vertical plane. The particles pass through another connector 115b to a third module 132a. Two sheath flows enter the third module 132a through ports 132a-1, 132a-2 positioned to each side (e.g. above and below) of the module channel and at least one of (and preferably both of) hydrodynamically fine tune the position and orientation of the particles in the horizontal plane. Together, sheath flows that enter the system through modules 144 and both 132a, 132b position the particles at a desired location within the microfluidic channel (e.g. confined to a central region of the microfluidic channel), and, in some embodiments, orient the particles in a desired orientation relative to the laboratory frame of reference (e.g., with one particle feature oriented vertically relative to the laboratory frame of reference). The oriented, positioned particles pass through a functional connector 115c that allows two functional steps (at least). First, the particles encounter a particle discrimination stage I which includes a function of discriminating the particles into two or more subsets. In this stage, the particles can be illuminated by a radiation source 112a (e.g. narrow band) configured to induce the particles to emit fluorescence. The fluorescence is transmitted to two optical detectors 116a, 116b positioned orthogonally to the direction of the microfluidic flow (e.g. above and to one side, in this embodiment) that transmit the fluorescence signal electronically to a controller/signal collector/signal processor system 118. As fluorescence from each particle is detected by the two detectors, system 118 determines whether to switch that particle into a different flow stream, based upon one or more features of the fluorescence signals (e.g. intensity, band shape, differences in the signals from each detector). If the decision is positive, that is, to switch the particle into a different flow stream, the electronic system 118 sends a signal to a radiation source 112b to induce switching to illuminate the particles after passing through a radiation beam shaping system 113a, as they flow through the functional connector/linker 115c, for a time to illuminate the particles so as to generate a force perpendicular to the axis of the flow stream and displace the particle horizontally (i.e., to one side or the other in microfluidic channel of the connector 115c. In the illustrated embodiment, the particles can be placed into three distinct, parallel flow streams. The particles then pass from the functional connector/linker 115c to module 122 in the three parallel flow streams within the same channel. The microfluidic T-junction in module 122 (see also module 400, FIGS. 4A-4B) allows one of the outer flow steams to be separated such that it branches off of the main flow stream containing the remaining two parallel, distinct particle flow streams and is directed perpendicularly to flow through the yet another functional connector 115d to module 110b. As the particles flow through the functional collector 115d, they encounter two functional steps. First, they are illuminated by a radiation source 112c (e.g. broadband) and imaged with an objective 113b and camera 113c. An electronic signal corresponding to the optical image of each particle is transmitted to system 118. As the image from each particle is detected, system 118 determines whether to switch that particle into a different flow stream, based upon one or more features of the particle image (e.g. size, optical density, morphology). If the decision is positive, that is, to switch the particle into a different flow stream, the electronic system 118 sends a signal to a radiation source 112d to induce switching to illuminate the particles after passing through a radiation beam shaping system 113d, as they flow through the functional connector/linker 115d, for a time to illuminate the particles so as to generate a force perpendicular to the axis of the flow stream and displace the particle vertically (i.e., to above or below the other in microfluidic channel of the connector). In this embodiment, the particles can be placed into two distinct, parallel flow streams within the functional connector 115b. The two parallel flow streams comprise two populations of particles that are collected in separate output flow streams, each leaving the microfluidic system through a different port 106a-1, 106b-1 (i.e., output port) in module 110. The particles that were not selected by 118 to be displaced in the first sorting stage associated with the first functional connector/linker, continue their linear flow along the main microfluidic channel through a connector 115e to module 110a. The two parallel flow streams remaining in the main channel comprise two populations of particles that are collected in separate output flow streams, each leaving the microfluidic system through a different port 106a-2, 106b-2 (i.e., output port) in module 110a. In such embodiments, a collection of particles can be sorted into four (4) populations that are collected in separate output flow streams, each leaving the microfluidic system through a different output module and a different port(s) (i.e., output port) in each output module 110a, 110b.

The plurality of modules may include, as shown in FIGS. 2A-2B, at least a first or initial module 200 (corresponding to module 110 in FIGS. 1A-1I) having at least one port 202 (input or output, depending upon use/function). While module 200 may be an initial module, it can also be arranged as a terminal or last module for collecting resultant products (particles, waste). Module 200 can include two (2) ports 202a, 202b, each of which can be connected to a source (not shown), or a collection reservoir (and/or the like) via a connection tube (which may be capillary sized). Such sources can be, for example, a sheath fluid reservoir/pump system, and particle fluid reservoir/pump system. Each of the ports may be configured to lead (or stem from) to a main, microfluidic channel 204. Channel 204 is preferably between 100-2000 microns in size (inner diameter), and may be circular or square in cross-section or any desired shape. Channel 204 includes an input/output 206, which is configured for connection to another (e.g., adjacent) module. The output 206 of the microfluidic channel can be sized so as to suit introduction of a functional connector (which can be a capillary tube). As noted above, the module of FIGS. 2A-2B may also be configured to operate as a final module, which may also be referred to as a collection module (see below). Thus, the ports 202a, 202b can be configured to outflow for example waste fluid (e.g., port 202a), and desired particles to collect (202b) or to collect two desired populations of output flows. The output 206 can thus also be an input to the module to receive a flow from another module.

Importantly, in any of the disclosed modules,—each port, and in some embodiments, the associated microfluidic channel, are configured to receive any one or more of: a connector (e.g., functional connector), a capillary tube, fibers optics, and a sensor(s).

The modular system may include at least one (e.g., second) module 300 (which is referred to as module 120 in FIGS. 1A-1C), embodiments of which are illustrated in FIGS. 3A-B, and FIGS. 4A-B (module 400, which is referred to as module 122 in FIGS. 1D-1I). Accordingly, modules 300/400 may comprise a cross junction module, which can comprise any combination of inputs to outputs, each with a microfluidic channel connected to each. The microfluidic channels are connected via a junction 306/406 within the module (in some embodiments, a junction is any portion in which at least two channels meet, and in still further embodiments, and portion in which at least two channels meet and which includes a narrower cross-section than at least one of the channels; see e.g., FIGS. 3A-9C). FIGS. 3A-3B illustrate a total of four (4) inputs/outputs 302, and FIGS. 4A-4B illustrate a combination of a total of three (3) inputs/outputs 402, which can be configured in any combination (e.g., one input, two outputs, two inputs, two outputs, and the like). The module of FIGS. 3A-3B, have at least one input connected to at least one output, via a microfluidic channel 304. Optional inputs/outputs 306 and 308 can also connect to the other inputs and outputs via a respective microfluidic channel and the junction 305. Similarly, the module of FIGS. 4A-4B, includes a total of three inputs and outputs, with two (2) inputs, 402 and 406, and only one (1) output 404, each connected to a microfluidic channel, all in fluid communication via junction 405.

Such junction modules have microfluidic channels with inputs/outputs sized or otherwise configured for at least one of capillary tube insertion (e.g., for fluid/particle flows), fiber optics insertion, and/or other functional connections. In some embodiments, the inputs/outputs/microfluidic tubes may be sized between 50-4000 microns. For example, in FIGS. 3A-3B, two of the opposed inputs/outputs can be used with fiber optics for transmitting and/or detecting light, while the two remaining opposed inputs/outputs, set perpendicular thereto, may be used as capillary tube (e.g.) for fluid/particle flow inputs and/or outputs. Similarly, in FIGS. 4A-4B, there may only be a single input/output (and corresponding microfluidic channel), which is configured to receive fiber optics, while the two opposed and orthogonal inputs/outputs are configured for fluid/capillary tube input/output.

FIGS. 5A-B are illustrations of one of the modules for a modular microfluidic system according to some embodiments of the present disclosure, where FIG. 5A illustrates a top view and FIG. 5B illustrates a perspective view thereof. This module 500 can be used as a “turning” module, to direct a flow in an orthogonal direction, or, may be used as terminus, and includes ports 502, associated microfluidic channels 504, and junction 506.

Other modules include (which may be initial, intermediary, or terminal modules), for example:

FIGS. 6A-6B illustrate a module 600 (corresponding to module 132 in FIGS. 1D-1I) which includes two (2) ports (source/collection) 602a, 602b, a port 602c (e.g., input port) and a port 602d (e.g., output port); the module may include a junction 605. Each port is connected to a microfluidic channel 604 (and which can meet at a junction 605). In some embodiments, sheath flow from the inputs 602a, 602b are used to focus particles (e.g., biological cells) in an existing flow in the microfluidic channel of the module.

FIGS. 7A-7B illustrates a module 700 (which corresponds to module 130 in FIGS. 1A-1C) which in two (2) ports 702a, 702b (e.g., source inputs) a port 702c (e.g., input) and port 702d (e.g., an output). The ports are connected to a microfluidic channel 704 (the configuration may also include junction 705). Additionally, the module 700 shown in FIGS. 7A-7B includes filter elements 707 which may be configured as pillar elements, with spaced apart circular regions or regions of any shape. In some embodiments, such a configuration filters contaminants within the fluid flow, and in some embodiments, may be used to reduce clumps of biological cells from entering a main flow.

FIGS. 8A-8C illustrate yet another module 800 (corresponding to module 142 in FIGS. 1D-F) for particle manipulation systems according to some embodiments of the present disclosure. The module of FIGS. 8A-8B, can be configured, in some embodiments to be an terminus/end or last module (which may also be referred to, in some embodiments, as a collection module) in a particular manipulation system. In many embodiments, the module is merely an input module. The module can include a microfluidic channel 804 with receives a flow from a prior module, and can include multiple ports 802a-d (inputs/outputs), as well as a junction 805 and filter element 807. One of skill in the art will appreciate that the module of FIGS. 8A-8B can also be configured for use in a first or initial module of a particular manipulation system (e.g., one or more ports being inputs, one or more ports being outputs). For example, ports 802a-c can be configured as input ports, to receive flows (e.g., particle fluid flows, sheath flows).

If configured as a collection module, module 800 the ports 802a, 802b, and 802c can collect, for example, material passed to the collection module from the at least one other module, and/or a first of the at least two output ports of the collection module can collect particles of interest received from the previous module(s), and a second of the at least two outputs of the collection module collects waste. FIG. 8C is an enlarged portion of the microfluidics channel 804 of the module 800. This figure shows an example of a micron-sized capillary tube inserted into the input 802d thereof.

The module of FIGS. 8A-8C can also be configured as an initial module, in which any of ports 802a and 802c can be configured as sheath flows, to focus particles in a particle flow originating from port 802b, with the combined flow existing out port 802d of the microfluidic channel 804.

FIGS. 9A-9B illustrate yet another module 900 (corresponding to module 144 in FIGS. 1G-I), which is similar to the module 800 of FIGS. 8A-B, but lacks a filter element. The module of FIGS. 9A-9B, can be configured, in some embodiments to be an input, or terminus/end or last module (which may also be referred to, in some embodiments, as a collection module) in a particular manipulation system. The module can include a microfluidic channel 904 with receives a flow from a prior module, and can include multiple ports 902a-d (inputs/outputs), as well as a junction 905. As noted above, one of skill in the art will appreciate that the module of FIGS. 9A-9B can also be configured for use in a first or initial module of a particular manipulation system (e.g., one or more ports being inputs, one or more ports being outputs). For example, ports 902a-c can be configured as input ports, to receive flows (e.g., particle fluid flows, sheath flows).

The system further includes at least one connector connecting pairs of modules (e.g., adjacent modules), each being preferably configured to carry a fluid flow (which may also contain particles), and which may also be configured to carry out at least one additional function. Each connector includes a lumen surrounded by a wall, where the lumen may be configured to flow at least particles contained in a fluid therethrough and between connected modules. Each connector also includes a first end in fluid communication with an output port of one of the connected modules, a second end in fluid communication with the input port of a remaining one of the connected modules. Such connectors are illustrated in FIGS. 1A-1I as reference 115.

In some embodiments, at least a first portion and/or another portion of the wall of at least one of the connectors is configured to at least one of receive light into the lumen and transmit light out of the lumen, or at least a first portion and/or another portion of the wall of the sorting connector is configured to at least one of receive and transmit light through the wall. Such portions may be made of, for example, glass, quartz, or a polymer, and are preferably configured to at least one of receive light into the lumen and transmit light out of the lumen. Such functionality can be configured for: receiving light so as to induce one or more fluorescence signals of material flowing within the lumen of the connector, transmitting one or more fluorescence signals generated by material flowing within the lumen of the connector through the wall, receiving light so as to induce a force or a torque upon material flowing within the lumen of the connector, transmitting light through the wall so as to induce one or more scattering signals by material flowing within the lumen of the connector, transmitting scattered light signals generated by material flowing within the lumen of the connector, transmitting light so as to illuminate at least one of the particles flowing within the lumen of the connector for imaging of the at least one of the particles, and transmitting light reflected off material flowing within the lumen for imaging the material.

Specific functions/functionality to be performed by a connector may also include, for example, at least one of: particle entry, particle sheathing, particle focusing, particle orienting, particle detecting, particle discrimination, particle sorting, and at least one of sample and particle collection.

For example in FIG. 1C, one of the connectors 115 includes a portion 117a (enlargement in the figure), in which light (e.g., fluorescence) generated by particles within the connector, passes out of at least one of the portions 117b, 117c. This light may be detected by an adjacently placed detector (e.g., imager).

In some embodiments, one or more connectors can be configured as a transparent capillary tube, such that a sample or particles within the system can be at least one of focused and oriented. For example, when the sample or particle is a cell (e.g., a sperm cell), the light passing through the transparent capillary can focus to the center of a channel within the microfluidic system and move the cell to adopt a particular orientation in the fluid flow.

Moreover, in some embodiments, the light passing through the transparent capillary connector tube can be configured to detect at least one difference between particles or discriminate between particles, via, for example, a fluorescent signal provided by the particle after being excited via a laser (for example). The light passing through the transparent capillary can also be configured to cause a change in direction of one more selected particles so as to sort the particles into a particular output based on detection information.

One or more modules, or the system as a whole, can be configured to provide hydrodynamic flow in multiple dimensions, where the dimensions can comprise three-dimensions. Moreover, in some embodiments, each module may comprise a plurality of sides, where input ports and output ports are configured for arrangement on any side. For example, all of the input ports can be arranged on a first side, all of the output ports can be arranged on a second side, one or more of the input ports can be arranged on a first side, and one or more of the output ports can be arranged on a second side. In some embodiments, at least one input port and at least one output port may be arranged on a first side, and at least one input port and at least one output port may be arranged on a second side.

In some embodiments, the microfluidic system(s) disclosed may be configured for use with features of the microfluidic system and methods disclosed in U.S. Pat. No. 9,784,663 (“the '633 patent), which is incorporated by reference in its entirety herein. In some embodiments, the systems of the '633 Patent provide an input source and at least two output sources and a plurality of stages for focusing, orienting, detecting, sorting and collecting a sample or particle. In some embodiments of the present disclosure, the separate modules or modules can be configured for use as the stages disclosed the '633 patent, and two or more may be connected by a transparent capillary (as discussed above).

In some embodiments, a microfluidic sorting method for sorting particles in a microfluidic system is provided, and includes, providing a modular, microfluidic sorting system for sorting particles in a microfluidic system, according to any of the disclosed embodiments, directing a sheath fluid flow from at least one first input source into at least one input port of at least one first module, directing a plurality of particles in a fluid from at least one second input source into at least one of the module channels within the sheath flow to create a particle flow, first passing the particle flow from one of the modules to another via at least one of the connectors, at least one of:
directing light into the at least one connector so as to illuminate material inside the connector;
at least one of monitoring and imaging light signals generated by the material within the lumen through the wall; and
directing light into at least one connector so as to induce at least one of a force and a torque on material flowing inside the connector;

The method also includes, second passing the particle flow from the one module into another module via at least one other connector, and at least one of:
ultimately directing material of interest received from at least one module through at least one connector to a collecting module and into a particle collection output port, and
ultimately directing waste material received from at least one module through at least one connector through a collecting module and into a waste collection output.

In some embodiments, a modular microfluidic particle method is provided, and includes interconnecting a plurality of modules configured to be interconnected in at least two arrangements, where each module and at least one connector includes at least one associated function. In some such embodiments, the associated function may be selected from the group consisting of particle entry, particle sheathing, particle focusing, particle orienting, particle detecting, particle discrimination, particle sorting and sample or particle collection.

### Particle Orientation & Delivery Tube (PODT) Aspects

As shown in FIGS. 10-22C, a particle orientation system which may be configured for at least positioning and/or orienting particles in a fluid flow within a microfluidic channel is provided.

As shown in FIG. 10, the orientation stage/system 1000, in some embodiments, comprises a sheath fluid tube or microfluidic channel 1002, and a PODT 1004. In the example provided in FIG. 10, the inner diameter of 1004 is 260 microns (ranging from 50 to 750 microns), and the outer diameter of 1004 is 464 microns (ranging from 100 to 1000 microns). The inner diameter of the channel, in this embodiment a capillary, is 700 microns (ranging from 100 to 1000 microns). The microfluidic channel may also comprise a chamber (see FIG. 22A), or, the microfluidic tube may direct a particle flow into a chamber. The sheath fluid enters the sheath fluid tube 1002 via flexible tube 1006. Particles 1008 (e.g., asymmetric particles, like, for example, sperm cells) enter the stage at 1010. The axis of flow is signified by reference number 1012.

Of particular relevance for the orientation stage/system, is the PTOD 1004, which is configured for delivering a particle-containing fluid comprising at least a plurality of particles within a fluid into the sheath fluid within the microfluidic channel or chamber. In some embodiments, at least one of the PODT, the microfluidic channel, and chamber wall include at least one structural feature or feature set which is configured to impart an orienting torque to one or more, and preferably, each of the plurality of particles within the sheath fluid.

Such a feature(s)/feature-set of the POTD, may include, for example, a chamfer, a cutaway or angled surface, an inserted or stamped/punched divider (e.g., placed within the central lumen of the PODT, and the like. A number of embodiments for this can be found in FIGS. 11-22C.

FIG. 11A illustrates a perspective view of the distal end of a PODT, which includes two opposed sets of chamfers, each set orthogonal to the other. As shown, a first set 1002A-1, 1002A-2 includes a lesser angled chamfer, than the other set 1004A-1, 1004A-2. Fluid flow direction is indicated by 1006.

FIGS. 11-11B illustrate various views of the distal end of another PODT, which is configured with a single set of opposed chamfers 1102-1, 1102-2. FIG. 11E is a side view of the distal end of the orientation tube, which while similar to those of FIGS. 11B-11D, includes a curved chamfer and not a straight chamfer. Depending upon the embodiment, such chambers may be: between 5-90 degrees, between 5-80 degrees, between 5-70 degrees, between 5-60 degrees, between 5-50 degrees, between 5-40 degrees, between 5-30 degrees, between 5-20 degrees, and between 5-10 degrees (and ranges therebetween). In some embodiments, the opposed chamfers equally divide the PODT so that they meet at the very end in the center of the PODT. In some embodiments, the chamfer is preferably approximately 40 degrees (e.g., within a few degrees thereof); the range of chamfer angles is 10 to 80 degrees relative to the axis of the direction of flow within the PODT. The PODT can be manufactured by laser micromachining such chamfers, and the resultant distal end (as well as, in some embodiments, a portion or all of the entire PODT) includes a post machining electropolish or other finishing process. FIG. 11E is similar to the embodiments of FIGS. 11B-11D, but includes a curved chamfer 1104-1, 1104-2 substantially similar to FIGS. 11B-11D, but includes a radius within the range of 10-75% of the outer diameter of the PODT.

FIGS. 12A-12C illustrate yet other embodiments of the PODT, with FIGS. 12A-12B illustrating a multiple angled cut 1202-1a/2a, 1202-1b/2b with the angle range for each cut from 10 to 80 degrees relative to the axis of flow within the PODT, and FIG. 12C including an opposed pair of angled cuts 1204a-d, in which one set (e.g., opposed), in some embodiments, is a different angle/dimension/configuration than the remaining set (e.g., opposed) within the range of 10 to 80 degrees relative to the axis of flow within the PODT. FIGS. 13A-13C represent various slit-style PODT configurations, including slits 1302a-b, and 1304 (which may include an opposed similar slit). The slit may encompass the entire outer diameter of the PODT or be as narrow as 10 microns.

FIGS. 14-21 illustrate various embodiments of the PODT (in perspective views), and associated flow stream simulations thereof, in both perspective, first side and top (i.e., or second side/orthogonal to the first side, views. The demonstrated exemplary flow simulations are according to the following specifications
Particle throughput (e.g., sperm cells) 100 particles per second
Microfluidic Channel diameter 700 microns
POTD Size 26 gauge (OD of 0.4626 mm, ID of 0.26 mm)
Total Volume flow rate—9×10⁻⁹ m³/s
Sheath/sample flow ratio—between 25:1 and 300:1
These parameters are exemplary only; the range of flow of particles can be from 100 particles per second to 50,000 particles per second depending upon the detailed architecture of the system. This corresponds to volume flow rates between 9.0×10⁻⁹ m³/s and 4.5×10⁻⁶ m³/s, under typical particle concentrations. FIG. 22 illustrates an embodiment that incorporates a larger, tapered chamber that exemplifies performance at such a higher flow rate.
The POTD size range for all embodiments is inner diameter of 50 to 1000 microns. The microfluidic channel range for all embodiments is inner diameter of 100 to 1000 microns. The range of maximum dimension for a microfluidic chamber, such as exemplified in FIG. 22, is from 300 to 10,000 microns for all embodiments.

The color coding of the flow lines in FIGS. 11-22 reflect absolute velocities of the fluid flow within the channel, with higher flow rates indicated by red lines and slower flow rates by blue lines. Only the sample flow is depicted; the sheath fluid flow is not shown for clarity. The flow velocities and relative flow velocities, the shapes of the velocity flow profiles, is an indicator of the asymmetric forces generated by the flows that acts to orient and/or position particles within the channel.

FIGS. 14A-14C, FIGS. 15A-15D, FIGS. 16A-16D, each correspond to particular embodiments of the PODT for an asymmetric compression of a focused flow stream, which, in some embodiments, facilitates orientation of, for example, asymmetrically shaped particles (e.g., sperm cells); the “A” figure corresponding to the perspective view of the distal end of the PODT, and the “B-D” drawings corresponding to the representations of the modeled flow (see drawing/figure brief descriptions, above). In such embodiments, as illustrated in FIGS. 14A-14D, the opposed chamfers 1401a-b (which meet approximately in the center of the PODT) includes an angle of about 40 degrees (range 10 to 80 degrees relative to the axis of flow within the PODT). The embodiments, as illustrated in FIGS. 15A-15D, which include an end-notch 1502 directly on the distal end of the PODT, includes a length of approximately 0.3 mm (10-200% of the outer diameter of the PODT) and a width of approximately 0.26 mm (range 10 microns to the inner diameter of the PODT) (centered on the center line of the PODT, in this embodiment however the notch can be located off of the central axis). FIGS. 16A-16D include an angled cut 1602a-b of approximately 0.5 mm by about 0.18 mm (with an angle range of 10 to 80 degrees relative to the axis of flow within the PODT, which also determines the range of lengths of the features relative to the PODT outer diameter; the depth of cut relative to the full (outer) radius of the PODT is from 10 to 90%).

FIGS. 17A-17D, correspond to embodiments of the PODT configured for repositioning a focused stream to an off-center positions. As such, the PODT of these embodiments include a single 20 degree chamfer 1702 on one side of the PODT which end on the centerline of the distal end (range 10 to 80 degrees relative to the axis of flow within the PODT).

FIGS. 18A-18D, correspond to embodiments of the PODT configured for splitting the particle stream. Specifically, the central flow of the stream is split into two, focused streams. This is effected by cutting a relatively narrow, long notch 1802a-b into the two opposite sides of the PODT which partitions the sample stream into two separated streams as illustrated.

FIGS. 19A-19D, correspond to embodiments of the PODT configured for an asymmetric compression and spread reduction of the flow stream by combining the embodiments of, for example, FIGS. 14A and 16A. In these embodiments, the opposed chamfers 1902a-b include an angle of approximately 80 degrees (range from 10 to 80 degrees relative to the axis of flow within the PODT) (which meet in the center of the distal end of the PODT), and opposed, angled notches 1904a-b cut proximate to the chamfered end. The angled notches, like those of FIG. 16A, are approximately 0.5 mm long (relative to the length of the PODT), and 0.18 mm deep (relative to the radius of the PODT) with ranges commensurate with those described for FIG. 16.

FIGS. 20A-20D, correspond to embodiments of the PODT for an asymmetric compression and repositioning of a particle flow stream, by combining the PODT embodiments illustrated in FIG. 16A (2002a-b) and FIG. 17A (2004). As such, the combination provides more complex functions to the focused stream, including, as illustrated in FIGS. 20A-20D, an asymmetrically compressed and repositioned off-center particle flow.

Similarly, FIGS. 21A-D, correspond to embodiments of the PODT for asymmetric compression and beam splitting, by combining the PODT embodiments illustrated in FIG. 14A (2102a-b) and FIG. 18A (2104a-b). This splits the particle flow stream into two streams, where each stream is subsequently asymmetrically compressed. In these embodiments, the opposed end chamfers are each is approximately 80 degrees and meet in the center of the PODT, and the stamped/punched divider corresponds to a sidewall portion of the PODT that is approximately 0.5 mm in length, by about 0.1 mm in width.

FIGS. 22A-C, correspond to an orientation stage with a PODT 2202 configured as that illustrated in FIG. 14A, arranged within a 2.5 mm diameter (maximum inner diameter) chamber 2204 (range from 0.5 to 10 mm) (which then is mated with a 0.24 mm inner diameter microfluidic channel 2206 (range from 50 to 1000 microns); the chamber configured to conically diverge 2208 to correspond to the 0.24 mm channel (the distal end of the chamber can be configured to fit within the channel size).

In any of the above embodiments:
a sheath fluid tube may configured to direct sheath fluid into the microfluidic channel or chamber (see e.g., FIG. 10);
the PODT may be inserted within at least one of the microfluidic channel or chamber, and/or the sheath fluid tube (see e.g., FIG. 10);
the at least one feature can be configured to generate an asymmetric pattern of laminar flow of the sheath fluid and fluid that contains the plurality of particles;
the design of the feature (e.g., structural feature) can be configured to effect a torque to orient particles at one or more stable points relative to a frame of reference comprising the microfluidic channel;
at least the distal end of the PODT is arranged at a particular location within the microfluidic channel or chamber in at least one location relative to a frame of reference comprising the microfluidic channel or chamber (see e.g., FIG. 10);
the plurality of particles comprise, for example, asymmetric particles, e.g., biological cells (e.g., sperm);

In some embodiments, a particle orientation method is provided which is configured for orienting a plurality of particles in a fluid contained within a microfluidic channel or chamber. The method includes providing a system or PODT according to any such embodiment disclosed herein, flowing a sheath fluid within at least one of the sheath tube and a microfluidic channel or chamber, flowing a fluid including a plurality of particles via the PODT into the sheath fluid, and orientating the plurality of particles within the fluid. Orientating is produced via the at least one structural feature included with or on at least one of the internal surface and the external surface of the POTD and the internal surface of the microfluidic channel or chamber.

In some embodiments, a particle orientation method is provided which is configured for orienting a plurality of particles in a fluid contained within a microfluidic channel. The method includes flowing a sheath fluid within at least one of a sheath tube and a microfluidic channel or chamber, flowing a fluid including a plurality of, for example, asymmetric particles via a particle orientation and delivery tube (“PODT”) into the sheath fluid, and imparting a torque to the plurality of particles so as to orient the particles at one or more stable points relative to a frame of reference comprising the microfluidic channel or channel.

Such embodiments (as described above, for example) may include at least one of (and in some embodiments, preferably a plurality of, and in some further embodiments, preferably include all of) the following features, structure, functionality, steps, and/or clarifications, yielding yet further embodiments of the present disclosure:
imparting a torque to the plurality of particles is accomplished via at least one feature included with or on at least one of the internal surface, and the external surface of the POTD and the internal surface of the microfluidic channel or chamber; and
prior to flowing a fluid including a plurality of, for example, asymmetric particles via a PODT into the sheath fluid, the method further comprises inserting PODT within at least one of the sheath tube and microfluidic channel;

### Spatial Patterning Aspects

Accordingly, FIGS. 23A-23C and FIGS. 24A-24C illustrate two (2) example of simple static or dynamic patterns according to some embodiments of the disclosure. As shown, 2302, 2402 is the direction of flow of particles in the microfluidic channel 2304, 2404. FIGS. 23A and 24A show views from the top of the channel 2304, FIGS. 23B and 24B show views from the side of the channel, and FIGS. 23C and 24C show views directly down the channel, with the flow of particles coming out of the page. In FIGS. 23A-23C and FIGS. 24A-24C, there are multiple beams 2306, 2406 incident upon the microfluidic channel propagating in the x-direction. In FIG. 23A-23C the multiple beams are parallel and separated along the z-axis of microfluidic flow 2302 while on FIG. 24A-24C the multiple beams are parallel and separated along the y-axis perpendicular to the directions of microfluidic flow in channel 2404 and beam propagation. The number of beams may vary from one (1) to many (e.g., up to 10 individual beams). The spatial shape of each beam may be simple or complex, may be the same for all of the multiple beams, the same for a sub-set of the beams, or different for all of the beams, and the shapes may be static or dynamic. If the multiple beams are dynamic, they may be simultaneously or independently dynamic.

FIG. 25A-25C illustrates an example of slightly more complex static or dynamic pattern, according to some embodiments, with the particle flow (2502 direction) and orientation of the views being the same as in FIGS. 23A-23C. In this example, multiple beams 2506 are incident on the channel 2404 propagating in the x direction, and the positions of their focal points within the channel 2504 are different (e.g., arranged linearly in the x-z plane at an angle relative to the direction of microfluidic flow 2502 in the channel). The spatial shapes of these beams may be simple or complex, may be the same for all of the multiple beams, the same for a sub-set of the beams, or different for all of the beams, and the shapes may be static or dynamic. If the multiple beams are dynamic, they may be simultaneously or independently dynamic.

FIGS. 26A-26F illustrate two (2) examples of more complex static or dynamic pattern according to some embodiments, with the particle flow 2602 and orientation of the views is the same as that shown in FIGS. 23A-23C. As shown, multiple beams 2606 are incident on the channel 2604 propagating in the x-direction. The multiple beams are parallel and separated along both the y- and z-axes to create a two-dimensional array of parallel beams. In FIGS. 26A-26C the beams are in a grid aligned with the x-z and y-z planes; in FIG. 26D-26F the beams are in a grid perpendicular to and rotated about the x-axis by an arbitrary angle. The spatial shapes of these beams may be simple or complex, may be the same for all of the multiple beams, the same for a sub-set of the beams, or different for all of the beams, and the shapes may be static or dynamic. The beams may be arranged in a highly symmetric grid or in a pattern that is less symmetrical, including randomly arranged. The number of beams may vary from three (3) to, e.g., thirty (30). The focal points of all beams may be in the same plane containing the particle flow (similar to FIGS. 23A-23C and FIGS. 24A-24C) or be in any arrangement of locations within the microfluidic channel (similar to FIG. 25A-25C). If the multiple beams are dynamic, they may be simultaneously or independently dynamic.

FIGS. 27A-27C illustrate an example of a more complex static or dynamic pattern according to some embodiments, with the particle flow 2702 and orientation of the views is the same as that shown in FIGS. 23A-23C. In this example, In this example, some beams propagate in a collinear arrangement perpendicular to particle flow along the x-direction, and some are do not propagate collinearly and are at an angle relative to the z-axis defined by the particle flow and the x-axis. In addition, for this example, the focal points of the multiple beams within the channel 2704 are different. As with the other figures, the spatial shapes of these beams may be simple or complex, may be the same for all of the multiple beams, the same for a subset of the beams, or different for all of the beams, and the shapes may be static or dynamic. The number of beams in each of the co-linear and off-axis patterns may vary from one (1) to many (e.g. up to 10 individual beams). If the multiple beams are dynamic, they may be simultaneously or independently dynamic.

FIG. 28A-28F illustrates two (2) examples of slightly more complex static or dynamic patterns, according to some embodiments, with the particle flow 2802 and orientation of the views is the same as in FIGS. 23A-23C. As shown in FIG. 28A-28C a single line-shaped beam is incident on the channel 2604 propagating in the x-z plane in the x-direction perpendicular to the flow of the particles (z). FIG. 28D-28F, a single line-shaped beam is incident on the channel 2804 propagating in the x-z plane at some non-perpendicular angle relative to axis defined by particle flow (z). The shapes may be static or dynamic. Examples of dynamic behavior might be intensity changes in different parts of the pattern or changing the angle of propagation relative to the axis defined by the particle flow.

FIG. 29 illustrates two examples of patterns that are dynamic in time by moving the point at which a beam 2908 interacts with the particles 2901 in the flow 2902 within the channel 2904, according to some embodiments, with the particle flow and orientation of the views is the same as in FIG. 23B. In FIG. 29A, the dynamic adjustment unit 2705 works in reflection, while in FIG. 29B, the dynamic adjustment unit 2705 works in transmission. The velocity of laser beam sweep may be linear, or may be according to some other function. The spatial shape of the laser beam may be simple or complex. The spatial shape of the laser beam may be static or dynamic as the beam position is changed.

FIGS. 30A-30C provides a physical example of a static pattern of five (5) parallel beams propagating in the x-direction separated spatially along the z-axis of microfluidic flow, analogous to FIG. 23A-C. FIG. 30A shows an image of the focal points of the individual beams, with the orientation as in FIG. 23B. FIG. 30B shows a quantification of the intensity profile of the pattern of five (5) beams in FIG. 30A. FIG. 30C is a transmission microscope image of the diffractive optical element designed and constructed to create the pattern from a single incident radiation source. The diffractive optical element in this example is a 0.1 degree spaced Dammann grating with five (5) spots and a −1000 mm Fresnel zone plate overlay for DC dephasing. The diffractive optical element is an amplitude-only mask fabricated on a chrome-on-glass substrate.

FIGS. 31A-D provides a physical example of a static pattern of five (5) beams with focal points at different points along the x-axis direction of propagation, similar to FIGS. 25A-25C without the displacement along the z-axis. FIG. 31A is computer generated Dammann grating pattern used to create this pattern of beams, and FIG. 31B is the transmission microscope image of the same Dammann grating as in FIG. 31A constructed as a binary-phase grating fabricated on poly(methyl methacrylate) with an SU8 photoresist. The pattern to be generated is illustrated in FIG. 31C. FIG. 31D show the intensities of the multiple beams along the x-propagation direction generated by the phase grating shown in FIG. 31B interacting with a single beam. Each panel in FIG. 31D is a brightness measurement along the x-axis direction measured at 0.5 mm increments. Reading from the top left panel, the small, bright spots in the first, third and fifth panels in the top row, and the second and fourth panels in the bottom row indicate the five (5) focal points separated by 1.0 mm in position along the x-axis.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be an example and that the actual parameters, dimensions, materials, and configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims, equivalents thereto, and any claims supported by the present disclosure, inventive embodiments may be practiced otherwise than as specifically described and claimed. Inventive embodiments of the present disclosure are directed to each individual feature, system, article, material, kit, method, and step, described herein. In addition, any combination of two or more such features, systems, articles, materials, kits, methods, and steps, if such features, systems, articles, materials, kits, methods, and steps, are not mutually inconsistent, is included within the inventive scope of the present disclosure. Embodiments disclosed herein may also be combined with one or more features, as well as complete systems, devices and/or methods, to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-80. (canceled)

81. A particle orientation system configured for at least positioning and/or orienting particles in a fluid flow within a microfluidic channel, the system comprising:
a microfluidic channel or chamber configured for at least one of receiving and flowing at least a sheath fluid, and
a particle orientation and delivery tube (“PODT”) configured for delivering a particle-containing fluid comprising at least a plurality of particles within a fluid into the sheath fluid within the microfluidic channel or chamber,
wherein at least one of the PODT, the microfluidic channel, and chamber wall includes at least one structural feature configured to impart an orienting torque to the plurality of particles within the sheath fluid.

82. The system of claim 81, wherein the at least one structural feature comprises at least one of a chamfer, cutaway or angled surface.

83. The system of claim 81, further comprising a sheath fluid tube configured to direct sheath fluid into the microfluidic channel or chamber.

84. The system of 81, wherein the PODT is inserted within at least one of the microfluidic channel or chamber, and the sheath fluid tube.

85. The system of claim 81, wherein the at least one structural feature is configured to generate an asymmetric pattern of laminar flow of the sheath fluid and fluid that contains the plurality of particles.

86. The system of claim 81, wherein the torque orients the particles at one or more stable points relative to a frame of reference comprising the microfluidic channel.

87. The system of claim 81, wherein the PODT includes a distal end which projects into the microfluidic channel or chamber.

88. The system of claim 81, wherein at least the distal end of the PODT is arranged at a particular location within the microfluidic channel or chamber in at least one location relative to a frame of reference comprising the microfluidic channel or chamber.

89. The system of claim 81, wherein the plurality of particles comprise asymmetric particles.

90. The system of claim 81, wherein the plurality of particles comprise cells.

91. The system of claim 81, wherein the plurality of particles comprise sperm.

92. The system of claim 81, wherein the system is configured as an orientation stage within a microfluidic system.

93. The system of claim 92, wherein the microfluidic system comprises a cell sorting system.

94. A particle orientation and delivery tube (“PODT”) configured for use in a particle orientation system, the PODT being configured to orient a plurality of particles within a fluid, wherein the PODT includes at least one structural feature with or on at least one of the internal surface, the external surface configured to impart a torque to the plurality of particles within the fluid.

95. The PODT of claim 94, wherein the feature comprises at least one of a chamfer, cutaway and angle.

96. The PODT of claim 94, wherein the PODT is inserted within at least one of a microfluidic channel or chamber, and a sheath fluid tube.

97. The PODT of claim 94, wherein the at least one feature is configured to generate an asymmetric pattern of laminar flow of the fluid to impart a torque upon the plurality of particles.

98. The PODT of claim 94, wherein the torque orients the particles at one or more stable points relative to a frame of reference comprising a microfluidic channel or chamber.

99. The PODT of claim 97, wherein the shape of the microfluidic channel or chamber contributes to generating the asymmetric pattern of laminar flow.

100. The PODT of claim 98, wherein the shape of the microfluidic channel or chamber contributes to generating the asymmetric pattern of laminar flow.
